SEQUENCE LISTING (1 sequence)

SEQ ID NO: 1 - DNA, 200 bp, synthetic construct
  source 1..200: mol_type = other DNA
gttttacaatttcataggaatcttgaatactgggccacactaatcactagatactttaaatatcatctttggatataagcacaatggagcttacctgtggtgacaccatgcaccactccctccttggttttggagcctacaaaaacaaattcaagacataagtctcaagctagccttaaattgctgattagctagttttc

(19) United States
(12) Patent Application Publication
(10) Pub. No.: US 2024/0034992 A1
(43) Pub. Date: Feb. 1, 2024

(54) DOPAMINERGIC NEURONS COMPRISING MUTATIONS AND METHODS OF USE THEREOF

(71) Applicant: FUJIFILM Cellular Dynamics, Inc., Madison, WI (US)

(72) Inventors: Deepika RAJESH, Madison, WI (US); Ali FATHI, Madison, WI (US); Jing LIU, Madison, WI (US); Kiranmayee BAKSHY, Madison, WI (US); Sarah DICKERSON, Madison, WI (US); Wayne POON, Madison, WI (US)

(73) Assignee: FUJIFILM Cellular Dynamics, Inc., Madison, WI (US)

(21) Appl. No.: 18/050,963

(22) Filed: Oct. 28, 2022

Related U.S. Application Data
(60) Provisional application No. 63/273,480, filed on Oct. 29, 2021.

(51) Int. Cl. C12N 5/0793; C12N 9/24; C12N 9/12; C07K 14/47

(52) U.S. Cl. CPC C12N 5/0619; C12N 9/2402; C12Y 302/01045; C12N 9/12; C12Y 207/11001; C07K 14/47; C12N 2506/45

(57) ABSTRACT

The present disclosure provides iPSC-derived dopaminergic neurons comprised disease-associated mutations. Further provided herein are methods for using the iPSC-derived dopaminergic neurons for the study of neuroinflammation, such as to identify novel targets, biomarkers, and therapeutic agents for the diagnosis, prognosis, and treatment of neurodegenerative diseases. Further provided herein are assays for studying neuroinflammation using the present cell culture models.

### PRIORITY CLAIM

This application claims the benefit of U.S. Provisional Patent Application No. 63/273,480, filed Oct. 29, 2021, which is incorporated herein by reference in its entirety.

This application contains a Sequence Listing XML, which has been submitted electronically and is hereby incorporated by reference in its entirety. Said XML Sequence Listing, created on Oct. 27, 2022, is named CDINP0111US.xml and is 1,933 bytes in size.

### BACKGROUND

### 1. Field

The present invention relates generally to the field of molecular biology and medicine. More particularly, it concerns compositions of neurons differentiated form induced pluripotent stem cells (iPSC) and methods of use thereof.

### 2. Description of Related Art

Human iPSC-derived dopaminergic neurons offer a developmentally and physiologically relevant in vitro model of human midbrain dopaminergic neurons that innervate different regions in the central nervous system (CNS) including the forebrain and striatum. Loss of dopaminergic neurons cause decreased dopamine levels in the CNS and result in neurodegenerative conditions including Parkinson's disease (PD). Dopaminergic neurons decline with age and are selectively vulnerable to oxidative stress generated by dopamine oxidation that increases with age. There is an unmet need for dopaminergic neurons with disease-associated mutations for disease modeling.

### SUMMARY

In some embodiments, the present disclosure provides an isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation.

In some aspects, the cell line has a GBA N370S mutation or LRRK2 G2019S mutation. In certain aspects, the cell line has a GBA N370S mutation, GBA L444P mutation, or GBA RecNil mutation. In particular aspects, the cell line has a GBA N370S mutation. In certain aspects, the cell line has a LRRK2 G2019S mutation, LRRK2 R1441G mutation, LRRK2 R1441C mutation, or LRRK2 I2020T mutation. In specific aspects, the cell line has a LRRK2 G2019S mutation. In certain aspects, the cell line has a SNCA A53T mutation, SNCA E46K mutation, SNCA duplication, or SNCA triplication. In some aspects, the cell line has a SNCA A53T mutation.

In some aspects, the iPSC of the iPSC-derived dopaminergic neuron is genetically engineered to comprise a GBA mutation, LRRK2 mutation, or SNCA mutation.

In some aspects, the iPSC of the iPSC-derived DA neuron cell line is an iPSC episomally reprogrammed from a donor with a neurodegenerative disease. In certain aspects, the iPSC of the iPSC-derived DA neuron is an iPSC episomally reprogrammed from a donor with Parkinson's disease.

In some aspects, the cell line is a human cell line. In certain aspects, the iPSC-derived dopaminergic neurons are midbrain dopaminergic neurons. In some aspects, the iPSC-derived dopaminergic neurons are end stage dopaminergic neurons which express FOXA2 and tyrosine hydroxylase (TH). In particular aspects, the iPSC-derived dopaminergic neurons have at least 50% increased transcript levels of TH. DDC, MAOA, and/or COMT as compared to DA neurons differentiated from iPSCs reprogrammed from a healthy donor without disease-associated mutations. In specific aspects, the iPSC-derived dopaminergic neurons have at least 30% increased release of dopamine in response to KCl stimulation as compared to DA neurons differentiated from iPSCs reprogrammed from a healthy donor without disease-associated mutations. In some aspects, the iPSC-derived dopaminergic neurons have increased cell death, mitochondrial stress, and alpha-synuclein protein aggregation as compared to DA neurons differentiated from iPSCs reprogrammed from a healthy donor without disease-associated mutations. In certain aspects, the iPSC-derived dopaminergic neurons comprising an SNCA mutation have decreased lysosomal GCase activity as compared to DA neurons differentiated from iPSCs reprogrammed from a healthy donor without disease-associated mutations. In certain aspects, the iPSC-derived dopaminergic neurons comprising an SNCA mutation have decreased lysosomal GCase activity as compared to DA neurons differentiated from iPSCs reprogrammed from a healthy donor without disease-associated mutations. In some aspects, the iPSC-derived dopaminergic neurons have deficits in intracellular calcium signaling as measured by RNA sequencing and calcium imaging. In particular aspects, the cell line is isogenic.

A further embodiment provides a kit comprising the cell line of the present embodiments (e.g., an isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation) in a suitable container.

In some aspects, the kit comprises an iPSC-derived DA neuron cell line comprising a GBA N370S mutation in a first container and an iPSC-derived DA neuron cell line comprising a LRRK2 G2019S mutation in a second container. In certain aspects, the kit further comprises an iPSC-derived DA neuron cell line that does not comprise a disease-associated mutation. In particular aspects, the iPSC of the iPSC-derived DA neuron cell line is an iPSC episomally reprogrammed from a healthy donor, such as a healthy donor that does not have a neurodegenerative disease. In some aspects, the kit further comprises an iPSC-derived DA neuron cell line engineered to express a disease associated mutation in a third container. In certain aspects, the disease associated mutation is a mutation in alpha-synuclein (SNCA). In some aspects, the mutation in SNCA is a missense point mutation, such as A53T.

In some aspects, the kit further comprises astrocytes, pericytes, brain microvascular endothelial cells, microglia, and/or neurons each in a suitable container. In certain aspects, the kit further comprises reagents for detecting the level of dopamine, GBA activity, neuronal maestro multi-electrode array (MEA) activity, and alpha-synuclein-mediated protein aggregation each in a suitable container. In further aspects, the kit further comprises reagents to measure TH, DDC, MAOA and/or COMT transcript levels. In certain aspects, the reagents are further defined as an enzyme-linked immunosorbent assay (ELISA) reagents. In some aspects, the kit further comprises an ELISA plate. In some aspects, the kit further comprises reagents to detect glucocerebrosidase (GCase) activity and/or perform calcium imaging.

Further provided herein is the use of a kit of the present embodiments (e.g., a kit comprising the cell line of the present embodiments (e.g., an isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation) in a suitable container) for the detection of a neurodegenerative disease. In some aspects, the neurodegenerative disease is Parkinson's disease.

Another embodiment provides the use of the kit of the present embodiments (e.g., a kit comprising the cell line of the present embodiments (e.g., an isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation) in a suitable container) for the screening of therapeutic agents for the treatment of a neurodegenerative disease.

In yet another embodiment, there is provides the use of the kit of the present embodiments (e.g., a kit comprising the cell line of the present embodiments (e.g., an isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation) in a suitable container) as a model for Parkinson's disease.

Another embodiment provides a culture comprising the iPSC-derived DA neurons comprising a GBA N370S mutation, a LRRK2 G2019S mutation, or a SNCA A53T mutation.

In some aspects, the culture comprises iPSC-derived DA neurons comprising a GBA N370S mutation. In certain aspects, the culture comprises iPSC-derived DA neurons comprising a LRRK2 G2019S mutation. In some aspects, the culture comprises iPSC-derived DA neurons comprising a SNCA A53T mutation.

In certain aspects, the cell culture is a two-dimensional (2D) culture. In some aspects, the media comprises DAPT. In particular aspects, the cells are cultured on a surface coated with an extracellular matrix protein. For example, the extracellular matrix is basement membrane extract (BME) purified from murine Engelbreth-Holm-Swarm tumor. In some aspects, the extracellular matrix protein is MATRIGEL®, GELTREX™, collagen, or laminin. In some aspects, the cell culture is a three-dimensional (3D) culture.

In some aspects, the cell culture further comprises astrocytes, pericytes, brain microvascular endothelial cells, microglia, and/or other types of neurons.

In certain aspects, the iPSCs are human. In some aspects, the culture is xeno-free, feeder-free, and/or conditioned-media free. In particular aspects, the media is defined media.

Further provided herein is the use of the culture of the present embodiments (e.g., a culture comprising the iPSC-derived DA neurons comprising a GBA N370S mutation, a LRRK2 G2019S mutation, or a SNCA A53T mutation) as a model of a neurodegenerative disease. In some aspects, the model comprises a culture with iPSC-derived DA neurons comprising a GBA N370S mutation, iPSC-derived DA neurons comprising a LRRK2 G2019S mutation, iPSC-derived DA neurons derived from a healthy donor, or iPSC-derived DA neurons engineered to express an SNCA A53T mutation.

Another embodiment provides a method for screening a therapeutic compound for treating a neurodegenerative disease comprising contacting a test compound with iPSC-derived DA neurons of the present embodiments or a culture of the present embodiments (e.g., a culture comprising the iPSC-derived DA neurons comprising a GBA N370S mutation, a LRRK2 G2019S mutation, or a SNCA A53T mutation) and measuring the functional activity, physiology, or viability of the cells.

In some aspects, an increase in functional activity indicates the test compound is capable of treating a neurodegenerative disease. In certain aspects, the method comprises measuring levels of dopamine. In some aspects, further comprising contacting the cell line with KCl. In some aspects, measuring functional activity comprises measuring glucocerebrosidase (GCase) activity. In certain aspects, an at least 10% increase in GCase activity identifies a candidate compound. In some aspects, the method further comprises performing calcium imaging assay to measure calcium oscillations. In some aspects, measuring calcium oscillations comprises measuring one or more of the measurements consisting of peak number, peak rate and area under the curve. In certain aspects, measured calcium oscillations can be associated with AHN neurons or mutant neurons. In some aspects, measuring calcium oscillations can be used for compound screening.

In certain aspects, measuring functional activity comprises measuring GBA activity, MEA activity, alpha-synuclein-mediated protein aggregation, and/or mitochondrial ROS levels. In some aspects, the levels of dopamine are measured by an ELISA assay, such as a competitive dopamine ELISA. In some aspects, measuring dopamine levels comprises measuring mRNA and/or protein levels of dopamine. In particular aspects, an increase of at least 30% or higher than 30 ng/mL of dopamine indicates a candidate compound.

In some aspects, the method further comprises measuring TH, DDC, MAOA and COMT transcript levels. In certain aspects, alpha-synuclein protein aggregation is measured by thioflavin staining and alpha synuclein expression.

In certain aspects, the neurodegenerative disease is Parkinson's disease.

In some aspects, an at least 1.5 fold increase in TH, DDC, MAOA, and/or COMT RNA expression identifies a candidate compound. In certain aspects, an increase in MEA activity identifies a candidate compound. In some aspects, an at least 30% increase in alpha-synuclein expression and aggregation and/or mitochondrial ROS identifies a candidate compound. In particular aspects, an at least 50% decrease in GBA activity identifies a candidate compound.

Another embodiment provides a method for screening for a neurodegenerative disease comprising contacting iPSC-derived DA neurons comprising a GBA N370S mutation or LRRK2 G2019S mutation with a sample.

In some aspects, the method further comprises contacting iPSC-derived DA neurons comprising a SNCA A53T mutation with said sample. In some aspects, the iPSC-derived DA neurons comprising the GBA N370S mutation and/or the iPSC-derived DA neurons comprising the LRRK2 G2019S mutation are cells according to the present embodiments. In some aspects, the sample is a patient sample. In certain aspects, the sample is a blood sample. In some aspects, the method further comprises detecting the level of dopamine. In certain aspects, increased levels of dopamine indicate the presence of a neurodegenerative disease. In some aspects, the neurodegenerative disease is Alzheimer's disease, Parkinson's disease, Huntington's disease, or multiple sclerosis.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1F: Generation and characterization of iPSCs for PD modelling. (FIG. 1A) Episomally reprogrammed iPSC 01279 was engineered to generate a heterozygous (HZ) SNCA A53T mutant line. The engineering is schematically described, showing the amino acid 53 change from alanine to threonine highlighted in yellow. (FIG. 1B) IPSC derived from a PD patient harboring the GBA N370S mutation. The amino acid change of asparagine 370 to a serine is highlighted in yellow. (FIG. 1C) IPSC derived from a PD patient harboring the mutation LRRK2 G2019S mutation is schematically described, showing the amino acid change of glycine 2019 to a serine highlighted in yellow. (FIGS. 1D-1F) Cytogenetic analysis on G-banded metaphase cells from each iPS cell line detailing a normal karyotype, SNCA A53T, GBA (N370S), and LRRK2 (G2019S).

FIGS. 2A-2C: Generation and characterization of dopaminergic neurons from apparently health normal (AHN) iPSCs, engineered isogenic iPSCs and PD patients for disease modelling. (FIG. 2A) A schematic representation of the differentiation process from iPSC to the dopaminergic neurons with the timeline and cytokines utilized throughout the process is shown. (FIG. 2B) Representative flow cytometry dot plots of the dopaminergic progenitors at day 17, expressing midbrain transcription factors FOXA2 and LMX1. (FIG. 2C) Representative flow cytometry dot plots of the dopaminergic neurons at the end of process expressing FOXA2 and tyrosine hydroxylase (TH) enzyme which is the enzyme needed for dopamine synthesis from tyrosine.

FIGS. 3A-3C: (FIGS. 3A-3B) Immunofluorescent staining of end stage DA neurons. (FIG. 3C) Quantified flow cytometric analysis of FOXA2 and TH proteins in dopaminergic neurons.

FIGS. 4A-4C: GBA and LRRK2 PD patient iPSC-derived DA neurons show aberrant mRNA and protein expression of enzymes involved in dopamine synthesis and degradation. (FIG. 4A) Quantification of TH, DDC, MAOA and COMT transcript levels in ANH, engineered and PD patient derived DA neurons. (FIG. 4B) Quantification of TH and DDC protein levels in ANH, engineered and PD patient derived DA neurons. (FIG. 4C) Quantification of TH and DDC protein based on the area under the curve for each protein and normalized to the MAPT expression. (*P-value<0.05, **P-value<0.01, ***P-value<0.001).

FIGS. 5A-5B: GBA and LRRK2 PD patient iPSC-derived DA neurons release more dopamine when stimulated with KCl. (FIG. 5A) Multiple lots of cryopreserved DA neurons from GBA, LRRK2, SNCA and AHN iPSCs were thawed and plated for 21 days and the levels of dopamine released by the cells were quantified using a competitive dopamine ELISA (Eagle Biosciences Cat. No. DOP31-KO1) following the manufacturer's instructions. The standard curve graphed in Graphpad PRISM is shown. (FIG. 5B) The dopamine release of DA neurons was compared to the dopamine released by AHN DA neurons at day 21 cultures in the HBSS buffer and upon KCL stimulation (*P-value<0.05, **P-value<0.01, ***P-value<0.001).

FIGS. 6A-6E: SNCA engineered iPSC and PD patient iPSC-derived DA neurons reveal higher cell death and mitochondrial stress. (FIG. 6A) Representative staining of end stage DA neurons with 1 μM YOYO-3 Iodide (dead cells) and Calcein AM (live cells) at different timepoints. (FIG. 6B) Multiple lots of cryopreserved DA neurons derived from GBA, LRRK2, SNCA and AHN iPSCs were thawed and plated. Percentage of live/dead cells was calculated based on the positive objects in the green and red channels. The data depicts live cells per well of 96 well plate (0.143 cm²). (FIG. 6C) Representative images of DA neurons stained with MitoSOX dye (left image) and overlay with the phase image (middle image) and co-stained with Calcein AM for live cells (right image) (FIGS. 6D-6E) The quantification of the MitoSOX signal intensity (Means±SD) and percentage of the MitoSOX positive cells in the disease neurons compared to the AHN neurons is shown. (*P-value<0.05, **P-value<0.01, ***P-value<0.001)

FIGS. 7A-7C: SNCA engineered iPSC and PD patient iPSC-derived DA neurons have significantly more aSyn protein aggregation. (FIG. 7A) Thioflavin (Th-T) staining was performed to reveal the aggregated aSyn on live DA neurons culture at 22 DIV after adding oligomer aSyn (4 μM/ml) for 24 hrs. (FIG. 7B) All the aSyn⁺ cells were counted and plotted against Th-T signal intensity in the individual neurons in different bins of expression. (FIG. 7C) Th-T images were quantified on DA neurons culture at 21 DIV treated with active oligomer aSyn for 24 hrs, 48 hrs or 72 hrs (n=4, One-way ANOVA with Dunnett's multiple comparisons test).

FIGS. 8A-8C: SNCA engineered iPSC and PD patient iPSC-derived DA neurons have greater aSyn aggregation. (FIG. 8A) Quantification of Alpha-Synuclein protein aggregation using M the Meso Scale Diagnostics (MSD) U-PLEX human alpha-Synuclein kit (MSD-K15). Analyte concentrations were determined from the ECL signals by backfitting to the calibration curve. (FIG. 8B-C) Multiple lots of cryopreserved DA neurons derived from GBA, LRRK2, SNCA and AHN iPSCs were assayed at 21 or 42 days in culture.

FIGS. 9A-9F: SNCA engineered iPSC and PD patient iPSC-derived DA neurons have less GCase activity. Multiple lots of cryopreserved DA neurons derived from GBA, LRRK2, SNCA and AHN iPSCs were thawed and plated for 14 days and the samples were harvested and submitted for RNASeq analysis as well as western blot analysis. The FPKM values of the GBA transcript were quantified (FIG. 9A). The amount of GBA protein in the lysate was quantified based on the area under the curve for each protein and normalized to the MAPT expression (FIG. 9B). Different concentrations of 4-Methylumbelliferyl-β-D-glucopyranoside (4-MU) (FIG. 9C) as the substrate and GCase specific inhibitor Conduritol B Epoxide (CBE) (FIG. 9D) were tested. 10 mM of 4-MU and 2 mM of CBE were found to be the optimal concentrations and used for the following GCase activity assay. Different concentrations of protein lysate were also tested for the GCase activity from AHN DA neurons (FIG. 9E). 5 ug of protein lysate was used to compare GCase activities among different DA neurons (FIG. 9F). (n=4, p<0.001 one-way ANOVA with Dunnett's test for mean comparisons). (*P-value<0.05, **P-value<0.01, ***P-value<0.001).

FIGS. 10A-10B: SNCA engineered iPSC and PD patient iPSC-derived DA neurons reveal differences in the evolvement of neural network activity. Multiple lots of cryopreserved DA neurons derived from GBA, LRRK2, SNCA and AHN iPSCs were thawed and plated on the 48-well Classic multi-electrode array (MEA) plate (8 wells per lot at a cell density of 120K cells per well) in complete BrainPhys media. Neuronal activity was quantified using the Axion MaestroMEA. The results revealed lower activity and network strength in GBA, LRRK2 and SNCA neurons compared to the AHN cells on day 35 raster plots (FIG. 10A) along with the quantification of burst percentage, network burst frequency and synchrony index over time (FIG. 10B).

FIGS. 11A-11C: RNAseq data analysis at day 14 post thaw. Transcripts of mutant DA neurons were plotted against the AHN DA neurons and showed a high correlation in the gene expression level with significantly differential transcripts highlighted with colors (FIG. 11A). Differential gene expression analysis was carried out for mutant samples compared to the AHN samples and the number of transcripts that changed significantly was summarized in the Venn diagram (FIG. 11B). Gene set enrichment and pathway analysis for transcripts that are differentially regulated in mutant DA neurons compared to the AHN DA neurons in DAVID database. Pathways highlighted in color were commonly modulated pathways in all mutant DA neurons (FIG. 11C).

FIGS. 12A-12C: Compound screening using panel of iPSC-derived DA neurons. (FIG. 12A) Effects of 12 compounds at three concentrations on Gcase activity in GBA and LRRK2 DA neurons with AHN as the control. (FIG. 12B) For combinatorial screen, 4 molecules were picked from initial screen showing improved GCase activity and combined in different ways to test with GBA cells. Single compounds were tested both for acute exposure (3 days) and chronic exposure (two weeks). Combinatorial screen showed that chronic treatment with 10 uM Ambroxol had the highest GCase activity in GBA cells. (FIG. 12C) This was further tested in LRRK2, SNCA and GBA in comparison to AHN. At day 21 post thaw, treatment with Ambroxol at 10 uM for two weeks significantly increased Gcase activities in all mutant neurons. (Dotted lines indicate the Gcase levels of the DMSO vehicle control, ***P-value<0.001.)

FIGS. 13A-13D: Calcium imaging of iPSC-derived DA neurons. Calcium oscillation traces of day 14 post plated DA neurons were captured using FDSS/μCELL instrument for 20 minutes. (FIG. 13A) Baseline readings of Ca oscillation in AHN and all three mutations after treatment with 10 uM Ambroxol for one week. Compared to the untreated AHN DA neurons, treatment with Ambroxol caused hyperexcitability in AHN, LRRK2 and SNCA DA neurons but rescued the peak number and rate in GBA mutant DA neurons. Calcium transient properties including (FIG. 13B) number of peaks, (FIG. 13C) peak rates per minute and (FIG. 13D) peak amplitude (average) were quantified for all wells (repeats) of each cell line using Waveform software.

### DESCRIPTION OF ILLUSTRATIVE EMBODIMENTS

In certain embodiments, the present disclosure provides human iPSC-derived dopaminergic neurons with disease-associated mutations. Further provided herein are use of these cells as developmentally and physiologically relevant in vitro models of human midbrain dopaminergic neurons that innervate different regions in the CNS including the forebrain and striatum.

Loss of dopaminergic neurons causes decreased dopamine levels in the CNS and results in neurodegenerative conditions including Parkinson's disease (PD). Dopaminergic neurons decline with age and are selectively vulnerable to oxidative stress generated by dopamine oxidation that increases with age. The present studies outline the generation and characterization of iPSC-derived dopaminergic neurons from two PD patients that inherited either the GBA (N370S) or LRRK2 (G2019S) mutation (GBA and LRRK2 lines are part of the Parkinson's Progression Markers Initiative (PPMI) iPS cell bank) as well as genetically engineered SNCA A53T iPSCs. End stage dopaminergic neurons derived from healthy as well PD donors were assessed for GBA activity, neuronal MEA activity, and alpha-synuclein-mediated protein aggregation. These results recapitulated the many features of PD in a dish. Specifically, the present studies demonstrated that GBA and LRRK2 derived DAs produced and released more dopamine compared to the AHN DAs. GBA and LRRK2 DAs also showed higher alpha-synuclein aggregation comparable to SNCA DAs. Moreover, all mutant DAs (GBA, LRRK2 and SNCA) showed lower GBA activity, and their neuronal network activity developed faster but then decreased over time compared to the AHN DAs.

Thus, the present panel of human iPSC-derived dopaminergic neurons carrying disease-specific mutations can be used for various in vitro applications to uncover mechanistic insights of dopaminergic neuronal degeneration and identification of novel therapeutic targets.

Accordingly, in certain embodiments, the present disclosure provides in vitro culture models for the study of neuroinflammation, such as to identify novel targets, biomarkers, and therapeutic agents for the diagnosis, prognosis, and treatment of neurodegenerative diseases, such as Parkinson's disease. In particular aspects, the present cells and models may be used for the detection of early onset of Parkinson's disease.

Further provided herein are assays for studying neuroinflammation using the present cell culture models. The outcome of the present model can be GBA activity, neuronal MEA activity, mitochondrial oxidative stress and membrane potential, lysozyme and autophagy activity and alpha-synuclein-mediated protein aggregation. These DA neurons derived from patient derived iPSC provide an in vitro tool to create a more accurate model to understand complex interactions between other cells, such as human microglia and astrocytes, in a 2D or 3D organoid system and mimic neurogenerative diseases. The cells produced by the present methods may be used for disease modeling, drug discovery, and regenerative medicine.

In some aspects, the present DA neurons may be used for early diagnosis of PD using the dopamine release assay or by measuring TH, DDC, MAOA and COMT transcripts and protein expression level in the neurons. The lysosomal enzyme glucocerebrosidase (GCase) activity assay could be used on both PD and Gaucher disease patient-derived DA neurons regardless of familial or sporadic nature of PD as an early diagnostic test and disease phenotype for screening drugs and compounds. Alpha-synuclein aggregation and mitochondrial ROS level also could be used as a disease readout for early diagnosis and for screening drugs and compounds.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

As used herein, a composition or media that is “substantially free” of a specified substance or material contains ≤30%, ≤20%, ≤15%, more preferably ≤10%, even more preferably ≤5%, or most preferably ≤1% of the substance or material.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, a “cell line” is an established cell culture derived from one cell or set of cells of the same type that will proliferate indefinitely under certain conditions. The cells of the cell line may comprise a uniform genetic makeup.

“Feeder-free” or “feeder-independent” is used herein to refer to a culture supplemented with cytokines and growth factors (e.g., TGFβ, bFGF, LIF, analogs or mimetics thereof) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

The term “extracellular matrix protein” refers to a molecule which provides structural and biochemical support to the surrounding cells. The extracellular matrix protein can be recombinant and also refers to fragments or peptides thereof. Examples include collagen and heparin sulfate.

“Adherent culture,” refers to a culture in which cells, or aggregates of cells, are attached to a surface.

“Suspension culture,” refers to a culture in which cells, or aggregates of cells, multiply while suspended in liquid medium.

A “three-dimensional (3-D) culture” refers to an artificially-created environment in which biological cells are permitted to grow or interact with their surroundings in all three dimensions. The 3-D culture can be grown in various cell culture containers such as bioreactors, small capsules in which cells can grow into spheroids, or non-adherent culture plates. In particular aspects, the 3-D culture is scaffold-free. In contrast, a “two-dimensional (2-D)” culture refers to a cell culture such as a monolayer on an adherent surface.

The term “aggregate promoting medium” means any medium that enhances the aggregate formation of cells without any restriction as to the mode of action.

The term “aggregates,” i.e., embryoid bodies, refers to homogeneous or heterogeneous clusters of cells comprising differentiated cells, partly differentiated cells and/or pluripotent stem cells cultured in suspension.

“Neurons” or “neural cells” or “neural cell types” or “neural lineage” may include any neuron lineage cells, and can be taken to refer to cells at any stage of neuronal ontogeny without any restriction, unless otherwise specified. For example, neurons may include both neuron precursor cells and/or mature neurons. “Neural cells” or “neural cell types” and “neural lineage” cells can include any neuronal lineage and/or at any stage of neural ontogeny without restriction, unless otherwise specified. For example, neural cells can include neuron precursor cells, glial precursor cells, mature neurons, and/or glia.

As used herein “midbrain DA neuronal precursor cells,” “mDA neuronal precursor cells,” and “mDA precursor cells” are used interchangeably and refer to neuronal precursor cells that express FoxA2, Lmx1, and EN1 (a midbrain-specific marker); but the cells do not express Nurr1. Midbrain DA neuronal precursor cells may express one or more of: GBX2, OTX2, ETV5, DBX1TPH2, TH, BARHL1, SLC6A4, PITX3, PITX2, GATA2, NR4A2, GAD1, DCX, NXK6-1, RBFOX3, KCNJ6, CORIN, CD44, SPRY1, FABP7, SLC17A7, OTX1, and/or FGFR3. mDA precursor cells may express select genes at distinct stages of differentiation.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

### II. IPSC-DERIVED DOPAMINERGIC NEURONS

A. Pluripotent Stem Cells

In certain embodiments, the present disclosure provides methods for the differentiation of dopaminergic neurons from pluripotent stem cells, such as iPSCs. The differentiation may be by methods known in the art or by the methods disclosed herein.

Although in theory a pluripotent stem cell can differentiate into any cell of the body, the experimental determination of pluripotency is typically based on differentiation of a pluripotent cell into several cell types of each germinal layer. In some embodiments, the pluripotent stem cell is an embryonic stem (ES) cell derived from the inner cell mass of a blastocyst. In other embodiments, the pluripotent stem cell is an induced pluripotent stem cell derived by reprogramming somatic cells. In some embodiments, the pluripotent stem cell is an embryonic stem cell derived by somatic cell nuclear transfer. The pluripotent stem cell may be obtained or derived from a healthy subject (e.g., a healthy human) or a subject with a disease (e.g., a neurodegenerative disease, Parkinson's disease, etc.).

Induced pluripotent stem (iPS) cells are cells which have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2006, 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF. In some preferred embodiments, the iPS cells are human iPS cells.

Based on cell characteristics, cells of the ES cell-like colonies are induced pluripotent stem cells. The induced pluripotent stem cells are morphologically similar to human ES cells, and express various human ES cell markers. Also, when grown under conditions that are known to result in differentiation of human ES cells, the induced pluripotent stem cells differentiate accordingly. For example, the induced pluripotent stem cells can differentiate into cells having neuronal structures and neuronal markers. It is anticipated that virtually any iPS cell or cell lines may be used with the present invention, including, e.g., those described in Yu and Thomson, 2008.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mouse cells are also known (Takahashi and Yamanaka, 2006). Induction of iPS cells typically requires the expression of or exposure to at least one member from the Sox family and at least one member from the Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

IPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by, e.g., by injecting approximately 0.5-10×10⁶ cells into the rear leg muscles of 8-12 week old male SCID mice. Teratomas develop that demonstrate at least one cell type of each of the three germ layers.

In certain aspects of the present invention, iPS cells are made from reprogramming somatic cells using reprogramming factors comprising an Oct family member and a Sox family member, such as Oct4 and Sox2 in combination with Klf or Nanog, e.g., as described above. The somatic cell may be any somatic cell that can be induced to pluripotency such as, e.g., a fibroblast, a keratinocyte, a hematopoietic cell, a mesenchymal cell, a liver cell, a stomach cell, or a R cell. In some embodiments, T cells may also be used as source of somatic cells for reprogramming (e.g., see WO 2010/141801, incorporated herein by reference).

Reprogramming factors may be expressed from expression cassettes comprised in one or more vectors, such as an integrating vector, a chromosomally non-integrating RNA viral vector (see U.S. application Ser. No. 13/054,022, incorporated herein by reference) or an episomal vector, such as an EBV element-based system (e.g., see WO 2009/149233, incorporated herein by reference; Yu et al., 2009). In a further aspect, reprogramming proteins or RNA (such as mRNA or miRNA) could be introduced directly into somatic cells by protein or RNA transfection (Yakubov et al., 2010).

Methods for preparing and culturing pluripotent stem cells can be found in standard textbooks and reviews in cell biology, tissue culture, and embryology, including teratocarcinomas and embryonic stem cells: Guide to Techniques in Mouse Development (1993); Embryonic Stem Cell Differentiation in vitro (1993); Properties and uses of Embryonic Stem Cells: Prospects for Application to Human Biology and Gene Therapy (1998), all incorporated herein by reference. Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995).

After somatic cells are introduced into or contacted with reprogramming factors, these cells may be cultured in a medium sufficient to maintain the pluripotency and the undifferentiated state. Culturing of induced pluripotent stem (iPS) cells can use various medium and techniques developed to culture primate pluripotent stem cells, embryonic stem cells, or iPS cells, for example as described in U.S. Pat. Publication 2007/0238170 and U.S. Pat. Publication 2003/0211603, and U.S. Pat. Publication 2008/0171385, which are hereby incorporated by reference. It is appreciated that additional methods for the culture and maintenance of pluripotent stem cells, as would be known to one of skill, may be used with the present invention.

In certain embodiments, undefined conditions may be used; for example, pluripotent cells may be cultured on fibroblast feeder cells or a medium that has been exposed to fibroblast feeder cells in order to maintain the stem cells in an undifferentiated state. Alternately, pluripotent cells may be cultured and maintained in an essentially undifferentiated state using defined, feeder-independent culture system, such as a TeSR medium (Ludwig et al., 2006a; Ludwig et al., 2006b) or E8 medium (Chen et al., 2011; PCT/US2011/046796). Feeder-independent culture systems and media may be used to culture and maintain pluripotent cells. These approaches allow human pluripotent stem cells to remain in an essentially undifferentiated state without the need for mouse fibroblast “feeder layers.” As described herein, various modifications may be made to these methods in order to reduce costs as desired.

Various matrix components may be used in culturing, maintaining, or differentiating human pluripotent stem cells. For example, collagen IV, fibronectin, laminin, and vitronectin in combination may be used to coat a culturing surface as a means of providing a solid support for pluripotent cell growth, as described in Ludwig et al. (2006a; 2006b), which are incorporated by reference in their entirety.

Matrigel™ may also be used to provide a substrate for cell culture and maintenance of human pluripotent stem cells. Matrigel™ is a gelatinous protein mixture secreted by mouse tumor cells and is commercially available from BD Biosciences (New Jersey, USA). This mixture resembles the complex extracellular environment found in many tissues and is used by cell biologists as a substrate for cell culture.

Methods may be provided to improve neural differentiation (in particular midbrain DA differentiation efficiency) efficiency of pluripotent stem cells. Differentiation of pluripotent stem cells can be induced in a variety of manners, such as in attached colonies or by formation of cell aggregates, e.g., in low-attachment environment, wherein those aggregates are referred to as embryoid bodies (EBs). The molecular and cellular morphogenic signals and events within EBs mimic many aspects of the natural ontogeny of such cells in a developing embryo. Methods for directing cells into neuronal differentiation are provided for example in U.S. Publn. No. 2012/0276063, incorporated herein by reference. More detailed and specific protocols for DA neuron differentiation are provided in PCT Publication No. WO2013/067362, incorporated herein by reference.

Embryoid bodies (EBs) are aggregates of cells derived from pluripotent stem cells, such as ES cells or iPS cells, and have been studied for years with mouse embryonic stem cells. In order to recapitulate some of the cues inherent to in vivo differentiation, three-dimensional aggregates (i.e., embryoid bodies) may be generated as an intermediate step. Upon the start of cell aggregation, differentiation may be initiated and the cells may begin to a limited extent to recapitulate embryonic development. Though they cannot form trophectodermal tissue (which includes the placenta), cells of virtually every other type present in the organism can develop. The present invention may further promote neural differentiation following aggregate formation.

Cell aggregation may be imposed by hanging drop, plating upon non-tissue culture treated plates or spinner flasks; either method prevents cells from adhering to a surface to form the typical colony growth. ROCK inhibitors or myosin II inhibitors may be used before, during or after aggregate formation to culture pluripotent stem cells.

Pluripotent stem cells may be seeded into aggregate promotion medium using any method known in the art of cell culture. For example, pluripotent stem cells may be seeded as a single colony or clonal group into aggregate promotion medium, and pluripotent stem cells may also be seeded as essentially individual cells. In some embodiments, pluripotent stem cells are dissociated into essentially individual cells using mechanical or enzymatic methods known in the art. By way of non-limiting example, pluripotent stem cells may be exposed to a proteolytic enzyme which disrupts the connections between cells and the culturing surface and between the cells themselves. Enzymes which may be used to individualize pluripotent stem cells for aggregate formation and differentiation may include, but are not limited to, trypsin, in its various commercial formulations, such as TrypLE, or a mixture of enzymes such as Accutase®. In certain embodiments, pluripotent cells may be added or seeded as essentially individual (or dispersed) cells to a culturing medium for culture formation on a culture surface.

For example, dispersed pluripotent cells may be seeded into a culturing medium. In these embodiments, a culturing surface may be comprised of essentially any material which is compatible with standard aseptic cell culture methods in the art, for example, a non-adherent surface. A culturing surface may additionally comprise a matrix component as described herein. In some embodiments, a matrix component may be applied to a culturing surface before contacting the surface with cells and medium.

Substrates that may be used to induce differentiation such as collagen, fibronectin, vitronectin, laminin, matrigel, and the like. Differentiation can also be induced by leaving the cells in suspension in the presence of a proliferation-inducing growth factor, without reinitiating proliferation (i.e., without dissociating the neurospheres).

In some embodiments, cells are cultured on a fixed substrate in a culture medium. A proliferation-inducing growth factor can then be administered to the cells. The proliferation inducing growth factor can cause the cells to adhere to the substrate (e.g., polyornithine-treated plastic or glass), flatten, and begin to differentiate into different cell types.

B. Dopaminergic Neurons

Dopaminergic neurons of the midbrain are the main source of dopamine (DA) in the mammalian central nervous system. Their loss is associated with one of the most prominent human neurological disorders, Parkinson's disease (PD). In some embodiments, dopaminergic neurons may be generated as described in WO2013067362; WO2013163228; WO2012080248; or WO2011130675.

In some embodiments, the following methods may be used to generate dopaminergic neurons from pluripotent stem cells such as embryonic stem cells or iPS cells. For example, in some embodiments, the methods of U.S. Patent Application 2012/0276063 may be used to generate neurons from pluripotent stem cells. For example, in some embodiments, bFGF and TGFβ may be excluded from a media (e.g., excluded from a defined media such as TeSR or Essential8 media) that is used to culture pluripotent cells such as iPS cells prior to the start of aggregate formation (while cells were still in adherent culture), then this may be used to promote neuronal differentiation of the pluripotent cells. In some embodiments, when iPS cells are “primed” in the absence of TeSR growth factors, i.e., cultured in any medium that does not have basic fibroblast growth factor (bFGF) and transforming growth factor β (TGFβ), for several days prior to aggregate formation, the cells can develop into the neural lineage with purity, rapidity and consistency. Other methods for making neurons include Zhang et al. (2013), U.S. Pat. No. 7,820,439, PCT Publn. No. WO 2011/091048, U.S. Pat. Nos. 8,153,428, 8,252,586, and 8,426,200. In some aspects, mono-SMAD or dual-SMAD inhibition may be used to differentiate iPSCs to DA neurons.

In some aspects, either a single BMP signaling inhibitor or a single TGF-β signaling inhibitor is used to inhibit SMAD signaling in methods to convert pluripotent cells (e.g., iPS cells, ES cells) into neuronal cells such as midbrain dopaminergic cells. For example, in some aspects, pluripotent cells are converted into a population of neuronal cells comprising midbrain DA neurons, wherein the differentiation occurs in a media comprising a single BMP signaling inhibitor. In some embodiments, the BMP inhibitor is LDN-193189, dorsomorphin, or DMH-1. Non-limiting examples of inhibitors of BMP signaling include dorsomorphin, dominant-negative BMP, truncated BMP receptor, soluble BMP receptors, BMP receptor-Fc chimeras, noggin, LDN-193189, follistatin, chordin, gremlin, cerberus/DAN family proteins, ventropin, high dose activin, and amnionless. In some embodiments, a nucleic acid, antisense, RNAi, siRNA, or other genetic method may be used for inhibiting BMP signaling. As used herein, a BMP signaling inhibitor may be referred to simply as a “BMP inhibitor.” The BMP inhibitor may be included in the differentiation media on days 1, 2, 3, 4, 5, 6, 7, 8, 9, 10, 11, 12, 13, 14, 15, 16, and/or day 17 of differentiation, or any range derivable therein (e.g., days 1-17, 1-16, 1-15, 2-15, etc.). In some embodiments, the BMP inhibitor is included in the differentiation media on all of days 1-17 of differentiation. Nonetheless, it is anticipated that it may be possible to exclude the BMP inhibitor from the differentiation media at certain times, e.g., on 1, 2, or 3 of the above days. In some embodiments, the BMP inhibitor is optionally not included in the differentiation media on days 11-17, and in some preferred embodiments the BMP inhibitor is included in the differentiation media on days 1-10.

In some aspects, pluripotent cells were differentiated using mono-SMAD methods for a period of about 360-456 hours, more preferably about 384-432 hours, to produce a culture of neural cells. In the mono-SMAD methods, a single SMAD inhibitor such as a single BMP signaling inhibitor or a single TGF-β signaling inhibitor is used to inhibit SMAD signaling in methods to convert pluripotent cells (e.g., iPS cells, ES cells) into neuronal cells such as midbrain dopaminergic cells. Generally, and in contrast to other dual-SMAD methods of differentiation, mono-SMAD differentiation methods utilize only a single SMAD inhibitor, and a second SMAD inhibitor is not included in the differentiation media. For example, in some aspects, pluripotent cells are converted into a population of neuronal precursor cells including midbrain DA neuronal precursor cells, wherein the differentiation occurs in a media comprising a single BMP signaling inhibitor.

In some embodiments, the BMP inhibitor is LDN-193189, dorsomorphin, DMH-1, or noggin. For example, cells can be cultured in a media comprising about 1-2500, 1-2000, or 1-1,000 nM LDN-193189 (e.g., from about 10 to 500, 50 to 500, 50 to 300, 50, 100, 150, 200, 250, 300, 500, 750, 1000, 1250, 1500, 1750, 2000, 2250, or about 2500 nM LDN-193189, or any range derivable therein). In some embodiments, cells can be cultured in a media comprising about 0.1 to 10 μM dorsomorphin (e.g., from about 0.1 to 10, 0.5 to 7.5, 0.75 to 5, 0.5 to 3, 1 to 3, 0.25, 0.5, 0.75, 1, 1.25, 1.5, 1.55, 1.6, 1.65, 1.7, 1.75, 2, 2.25, 2.5, 2.75, 3, or about 2 μM dorsomorphin, or any range derivable therein). In some embodiments, cells can be cultured in a media comprising about 1 μM DMH-1 (e.g., about 0.2-8, 0.5-2, or about 1 μM DMH-1, or any range derivable therein). As shown in the below examples, LDN-193189, dorsomorphin, and DMH-1 were all successfully used in mono-SMAD inhibition methods to produce midbrain dopaminergic neurons from iPS cells.

In some aspects, a TGFβ inhibitor may be used to inhibit SMAD as a mono-SMAD inhibitor to generate midbrain dopaminergic neurons from pluripotent cells such as iPS cells. For example, in some embodiments, the differentiation media comprises at least a first TGFβ signaling inhibitor. Non-limiting examples of inhibitors of TGFβ signaling include A-83-01, GW6604, IN-1130, Ki26894, LY2157299, LY364947 (HTS-466284), A-83-01, LY550410, LY573636, LY580276, NPC-30345, SB-431542, SB-505124, SD-093, Sm16, SM305, SX-007, Antp-Sm2A, and LY2109761. For instance, the TGFβ inhibitor in a differentiation media may be SB431542. In some aspects, cells are cultured in a media comprising about 0.1 to 100 μM SB431542 (e.g., between about 1 to 100, 10 to 80, 15 to 60, 20-50, or about 40 μM SB431542). As used herein, a TGFβ signaling inhibitor, including a TGFβ receptor inhibitor, may be referred to simply as a “TGFβ inhibitor.” In some embodiments, a TGFβ inhibitor is not included in the differentiation media. In some embodiments, a TGFβ inhibitor (e.g., SB431542) be included in a differentiation media on days 1-3, or 1, 2, 3, and/or day 4 as the mono-SMAD inhibitor. As shown in the below examples, in some embodiments, a BMP inhibitor is used as the mono-SMAD inhibitor, since as these compounds were observed to produce superior differentiation of pluripotent cells into midbrain DA neurons, as compared to use of a TGFβ inhibitor.

In some aspects, a MEK inhibitor is included in a differentiation media, e.g., in combination with the BMP inhibitor or mono-SMAD inhibitor to produce midbrain dopaminergic neurons from pluripotent cells such as iPS cells. In some embodiments, the MEK inhibitor is PD0325901. Non-limiting examples of MEK inhibitors that could be used include PD0325901, trametinib (GSK1120212), selumetinib (AZD6244), pimasertib (AS-703026), MEK162, cobimetinib, PD184352, PD173074, BIX 02189, AZD8330 and PD98059. For example, in some embodiments, the method comprises culturing the cells in the presence of between about 0.1 and 10 μM (e.g., between about 0.1 and 5; 0.5 and 3 or 0.5 and 1.5 μM) of the MEK inhibitor, such as PD0325901. In some embodiments, cells are contacted with the MEK inhibitor (e.g., PD0325901) on day 3, 4, 5, or days 3-5 of the differentiation.

In some embodiments, midbrain DA neuronal precursor cells may be produced by a method comprising: obtaining a population of pluripotent cells; differentiating the cells into a neural lineage cell population in a medium comprising a MEK inhibitor (e.g., PD0325901), wherein the medium does not contain exogenously added FGF8b on day 1 of the differentiation; and further differentiating cells of the neural lineage cell population to provide an enriched population of midbrain DA neurons. In some embodiments, it has been observed that inclusion of FGF8 (e.g., FGF8b) in the differentiation media on day 1 can, in some instances, impede or prevent differentiation of the cells into midbrain DA neuronal precursor cells. In some embodiments, FGF8 may optionally be included in a differentiation media on later days of differentiation such as, e.g., days 9, 10, 11, 12, 13, 14, 15, 16, 17, or any range derivable therein, e.g., preferably wherein contact of pluripotent cells is initiated with the single SMAD inhibitor in a differentiation media on day 1.

In some aspects, a Wnt activator (e.g., a GSK3 inhibitor) is included in a differentiation media, e.g., in combination with the BMP inhibitor or mono-SMAD inhibitor to generate midbrain dopaminergic neuronal precursor cells from pluripotent cells such as iPS cells. In some embodiments, pluripotent cells into a population of neuronal cells comprising midbrain DA neurons, wherein the differentiation is in a media comprising at least a first activator of Wnt signaling.

A variety of Wnt activators or GSK3 inhibitors may be used in various aspects of the present disclosure. For example, the activator of WNT signaling can be a glycogen synthase kinase 3 (GSK3) inhibitor. Non-limiting examples of GSK3 inhibitors include NP031112, TWS119, SB216763, CHIR-98014, AZD2858, AZD1080, SB415286, LY2090314 and CHIR99021. In some embodiments, pluripotent cells are contacted with a single SMAD inhibitor that is not SB415286. In some embodiments, the activator of Wnt signaling is CHIR99021. Thus, in some aspects, a culture media for use according to the embodiments comprises from about 0.1 to about 10 μM CHIR99021 (e.g., between about 0.1 to 5, 0.5 to 5, 0.5 to 3, from greater than about 1.25 to 2.25, about 1.25, 1.5, 1.55, 1.65, 1.7, 1.75, 1.8, 1.9, 2.0, or about 1.75 μM CHIR99021, or any range derivable therein). In some preferred embodiments, about 1.6-1.7 μM, or about 1.65 μM of CHIR99021 is used.

In some aspects, an activator of Sonic hedgehog (SHH) signaling is included in a differentiation media, e.g., in combination with the BMP inhibitor or mono-SMAD inhibitor to generate midbrain dopaminergic neurons from pluripotent cells such as iPS cells. In some embodiments, the Sonic Hedgehog activator is Sonic Hedgehog (Shh) or a mutant Shh. The Shh can be, e.g., a human or mouse protein or it may be derived from a human or mouse Shh. For example, in some embodiments, the Shh is a mutant mouse Shh protein such as mouse C25II Shh or human C24II Shh. In some embodiments, the differentiation media comprises both Shh (e.g., C25II Shh) and a small molecule activator of SHH such as, e.g., purmorphamine. Without wishing to be bound by any theory, the Shh and/or activator of Sonic Hedgehog may promote neural floor plate differentiation.

Cultures of neuronal cell types that are derived from pluripotent cells, including iPS cells, are also commercially available and may be purchased. For example, iCell® Neurons, iCell® DopaNeurons, and iCell® Astrocytes are derived from human iPS cells and may be purchased from Cellular Dynamics International (Madison, Wisconsin). iCell® Neurons are human induced pluripotent stem cell (iPSC)-derived neurons that exhibit biochemical, electrophysiological, and pathophysiological properties characteristic of native human neurons. Due to their high purity, functional relevance, and ease of use, iCell® Neurons represent a very useful in vitro test system for neurobiology interrogations in basic research and many areas of drug development.

In some embodiments, a defined media (i.e., a media that does not contain tissue, feeder cells, or cell-conditioned media) may be used to produce neurons or astrocytes from pluripotent cells such as iPS cells.

Neural cells can be characterized according to a number of phenotypic criteria. The criteria include but are not limited to microscopic observation of morphological features, detection or quantification of expressed cell markers, enzymatic activity, neurotransmitters and their receptors, and electrophysiological function.

In some embodiments, the model may be provided in a microfluidic device. Various microfluidic device configurations useful for the support of cells, including in the form of in vitro blood vessel models, are known in the art. See, e.g., US 2011/0053207 and US 2014/0038279, which are incorporated by reference herein. In general, a microfluidic device comprising the blood brain barrier model as taught herein may comprise a chamber so dimensioned to accept the blood brain barrier model therein such that the endothelial cell layer and neuronal cell layer define a boundary between a first chamber or opening in fluid contact with the endothelial cell layer of the model, and a second chamber or opening in fluid contact with the neuronal cell layer of the model. The fluid may be a liquid such as a media or a buffer. The device may further comprise a fluid inlet and fluid outlet for each chamber, fluid reservoirs (e.g., media reservoirs) connected therewith, etc.

In some embodiments, the cells used in the cell cultures are generated from iPS cells that were generated from cells obtained from a healthy donor. In other embodiments, the donor has a disease. For example, in some embodiments the donor has a disease such as a neurological or neurodegenerative disease such as, e.g., epilepsy, autism, attention deficit-hyperactivity disorder (ADHD), amyotrophic lateral sclerosis (ALS), Charcot-Marie-Tooth (CMT), Huntington's disease, familial epilepsy, schizophrenia, familial Alzheimer's disease, Friedreich ataxia, spinocerebellar ataxia, spinal muscular atrophy, hereditary spastic paraparesis, leukodystrophies, phenylketonuria, Tay-Sachs disease, Wilson disease, an addiction disorder, depression, or a mood disorder. The disease may be a genetic disease or an increased genetic susceptibility to a particular neurological disease. Further provided herein are assays for studying neuroinflammation using the present cell culture models.

The cells are generally seeded in an appropriate culture vessel, such as a tissue culture plate, such as a flask, 6-well, 24-well, or 96-well plate. A culture vessel used for culturing the cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

C. Gene Disruption

In certain aspects, SNCA, GBA, or LRRK2 gene expression, activity or function is disrupted in cells, such as PSCs (e.g., ESCs or iPSCs). In some embodiments, the gene disruption is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the disruption can be effected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the disruption of the expression, activity, and/or function of the gene is carried out by disrupting the gene. In some aspects, the gene is disrupted so that its expression is reduced by at least at or about 20, 30, or 40%, generally at least at or about 50, 60, 70, 80, 90, or 95% as compared to the expression in the absence of the gene disruption or in the absence of the components introduced to effect the disruption.

In some embodiments, the disruption is transient or reversible, such that expression of the gene is restored at a later time. In other embodiments, the disruption is not reversible or transient, e.g., is permanent.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene disruption is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct. In particular aspects, the siRNA suppresses both wild-type and mutant protein translation from endogenous mRNA.

In some embodiments, the disruption is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 2005/0064474; 2006/0188987 and 2007/0218528, incorporated by reference in their entireties herein, for details regarding fusions of DNA-binding domains and nuclease cleavage domains. In some aspects, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some aspects, disruption of SNCA is carried out by contacting a first target site in the gene with a first ZFP, thereby disrupting the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains.

In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

In some embodiments, ZFNs target a gene present in the engineered cell. In some aspects, the ZFNs efficiently generate a double strand break (DSB), for example at a predetermined site in the coding region of the gene. Typical regions targeted include exons, regions encoding N terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. See, U.S. Patent Publication No. 2011/0301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, 1998) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. A library of TALENs targeting 18,740 human protein-coding genes has been constructed. Custom-designed TALE arrays are commercially available through Cellectis Bioresearch (Paris, France), Transposagen Biopharmaceuticals (Lexington, KY, USA), and Life Technologies (Grand Island, NY, USA).

In some embodiments the TALENs are introduced as trans genes encoded by one or more plasmid vectors. In some aspects, the plasmid vector can contain a selection marker which provides for identification and/or selection of cells which received said vector.

3. RGENs (CRISPR/Cas Systems)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

D. Differentiation Media

Cells can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, pH indicators, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully-defined and feeder-free media.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3-thioglycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

A differentiation medium according to certain aspects of the present disclosure can be prepared using a medium to be used for culturing animal cells as its basal medium. In some embodiments, a differentiation medium is used to differentiate pluripotent cells into midbrain dopaminergic neuronal precursor cells (e.g., D17 cells) using only a single BMP inhibitor or a single TGF-beta inhibitor. For example, a differentiation medium used to promote differentiation of pluripotent cells (e.g., into midbrain dopaminergic precursor cells) may comprise a single BMP inhibitor (such as LDN-193189 or dorsomorphin; e.g., on days 1-17 of differentiation; an activator of Sonic hedgehog (SHH) signaling (such as purmorphamine, human C25II SHH, or mouse C24II SHH; e.g., on days 1-6, 2-7, or 1-7); an activator of Wnt signaling (such as a GSK inhibitor, e.g., CHIR99021; e.g., on days 2-17 or 3-17) and/or a MEK inhibitor (such as PD0325901; e.g., on days 2-4 or 3-5). In some embodiments, a single TGFβ inhibitor (such as SB-431542; e.g., on days 1-4) may be used instead of the single BMP inhibitor; however, in some embodiments a single BMP inhibitor may result in superior differentiation of cells into FOXA2+/LMX1A⁺, cells as compared to use of a single TGF-β inhibitor. In some embodiments, FGF-8 (e.g., FGF-8b) is not included in differentiation media on the first day or days 1, 2, 3, 4, 5, 6, 7, 8, 9, or 10, or any combination thereof (e.g., days 1-8); for example, in some embodiments, FGF-8 is included in the differentiation media on days 9, 10, 11, 12, 13, 14, 15, 16, and 17, or any combination thereof. In various embodiments, the differentiation media may contain TGFβ and bFGF, or, alternately, the differentiation media may be essentially free of TGFβ and bFGF.

In certain aspects, a method of differentiation according to the embodiments involves passage of cell through a range of media conditions for example cells are cultured
in adherent culture in a medium comprising: a single BMP inhibitor (or a TGFβ inhibitor); an activator of Sonic hedgehog (SHH) signaling; and an activator of Wnt signaling;
in suspension in a medium comprising a single BMP inhibitor (or a TGFβ inhibitor); an activator of SHH signaling; and an activator of Wnt signaling, wherein cell aggregates are formed;
in adherent culture in a Neurobasal medium comprising B27 supplement, L-glutamine, BDNF, GDNF, TGFβ, ascorbic acid, dibutyryl cAMP, and DAPT, (and, optionally, lacking exogenously added retinol or retinoic acid) for maturation.

As the basal medium, any chemically defined medium, such as Eagle's Basal Medium (BME), BGJb, CMRL 1066, Glasgow MEM, Improved MEM Zinc Option, Iscove's modified Dulbecco's medium (IMDM), Medium 199, Eagle MEM, uMEM, DMEM, Ham, RPMI 1640, and Fischer's media, variations or combinations thereof can be used, wherein TGFβ and bFGF may or may not be included.

In further embodiments, the cell differentiation environment can also contain supplements such as B-27 supplement, an insulin, transferrin, and selenium (ITS) supplement, L-Glutamine, NEAA (non-essential amino acids), P/S (penicillin/streptomycin), N2 supplement (5 μg/mL insulin, 100 μg/mL transferrin, 20 nM progesterone, 30 nM selenium, 100 μM putrescine (Bottenstein, and Sato, 1979 PNAS USA 76, 514-517) and/or β-mercaptoethanol (β-ME). It is contemplated that additional factors may or may not be added, including, but not limited to fibronectin, laminin, heparin, heparin sulfate, retinoic acid.

Growth factors may or may not be added to a differentiation medium. In addition or in place of the factors outlined above, growth factors such as members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) family antagonists may be employed at various steps in the process. In some embodiments, FGF-8 is included in a differentiation media as described herein. Other factors that may or may not be added to the differentiation media include molecules that can activate or inactivate signaling through Notch receptor family, including but not limited to proteins of the Delta-like and Jagged families as well as gamma secretase inhibitors and other inhibitors of Notch processing or cleavage such as DAPT. Other growth factors may include members of the insulin like growth factor family (IGF), the wingless related (WNT) factor family, and the hedgehog factor family.

Additional factors may be added in an aggregate formation and/or differentiation medium to promote neural stem/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor. Neural cultures that are terminally differentiated to form post-mitotic neurons may also contain a mitotic inhibitor or mixture of mitotic inhibitors including but not limited to 5-fluoro 2′-deoxyuridine, Mitomycin C and/or cytosine β-D-arabino-furanoside (Ara-C).

The medium can be a serum-containing or serum-free medium. The serum-free medium may refer to a medium with no unprocessed or unpurified serum and accordingly, can include media with purified blood-derived components or animal tissue-derived components (such as growth factors). From the aspect of preventing contamination with heterogeneous animal-derived components, serum can be derived from the same animal as that of the stem cell(s). In some embodiments, the medium is a defined medium, and the medium does not contain serum or other animal tissue-derived components (such as irradiated mouse fibroblasts or a media that has been conditioned with irradiated fibroblast feeder cells).

The medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. For example, an alternative to serum may be prepared by the method disclosed in International Publication No. 98/30679. Alternatively, commercially available materials can be used for more convenience. The commercially available materials include knockout Serum Replacement (KSR) and Chemically-defined Lipid concentrate (Gibco).

The medium can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5, or 0.01, 0.02, 0.03, 0.04, 0.05, 0.1, 0.2, 0.5, 0.8, 1, 1.5, 2, 2.5, 5, 7.5, 10 mM or any intermediate values, but the concentration is particularly not limited thereto as long as it is appropriate for culturing the stem cell(s).

In some embodiments, pluripotent stem cells are cultured in a medium prior to aggregate formation to improve neural induction and floor plate patterning (e.g., prior to being dissociated into single cells or small aggregates to induce aggregate formation). In certain embodiments of the invention, the stem cells may be cultured in the absence of feeder cells, feeder cell extracts and/or serum.

E. Culture Conditions

A culture vessel used for culturing the cell(s) can include, but is particularly not limited to: flask, flask for tissue culture, spinner flask, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the cells therein. The cells may be cultured in a volume of at least or about 0.2, 0.5, 1, 2, 5, 10, 20, 30, 40, 50, 100, 150, 200, 250, 300, 350, 400, 450, 500, 550, 600, 800, 1000, 1500 mL, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel surface can be prepared with cellular adhesive or not depending upon the purpose. The cellular adhesive culture vessel can be coated with any substrate for cell adhesion such as extracellular matrix (ECM) to improve the adhesiveness of the vessel surface to the cells. The substrate used for cell adhesion can be any material intended to attach stem cells or feeder cells (if used). Non-limiting substrates for cell adhesion include collagen, gelatin, poly-L-lysine, poly-D-lysine, poly-L-ornithine, laminin, vitronectin, and fibronectin and mixtures thereof, for example, protein mixtures from Engelbreth-Holm-Swarm mouse sarcoma cells (such as Matrigel™ or Geltrex) and lysed cell membrane preparations (Klimanskaya et al., 2005). In some embodiments, the cellular adhesive culture vessel is coated with a cadherin protein, e.g., epithelial cadherin (E-cadherin).

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 7%, or any range derivable therein. The oxygen tension can be at least or about 1, 5, 8, 10, 20%, or any range derivable therein.

An adhesion culture may be used in certain aspects. If desired, the cells can be cultured in the presence of feeder cells. In the case where the feeder cells are used, stromal cells such as fetal fibroblasts can be used as feeder cells (for example, refer to; Manipulating the Mouse Embryo A Laboratory Manual (1994); Gene Targeting, A Practical Approach (1993); Martin (1981); Evans et al. (1981); Jainchill et al., (1969); Nakano et al., (1996); Kodama et al. (1982); and International Publication Nos. 01/088100 and 2005/080554). In some embodiments, feeder cells are not included in the cell culture media, and cells may be cultured using defined conditions.

In other aspects, a suspension culture may be used. Suspension cultures that may be used include a suspension culture on carriers (Fernandes et al., 2007) or gel/biopolymer encapsulation (U.S. Patent Publication No. 2007/0116680). Suspension culture of stem cells generally involves culture of cells (e.g., stem cells) under non-adherent conditions with respect to the culture vessel or feeder cells (if used) in a medium. Suspension cultures of stem cells generally include dissociation cultures of stem cells and aggregate suspension cultures of stem cells. Dissociation cultures of stem cells involve culture of suspended stem cells, such as single stem cells or those of small cell aggregates composed of a plurality of stem cells (for example, about 2 to 400 cells). When the dissociation culture is continued, the cultured, dissociated cells normally form a larger aggregate of stem cells, and thereafter an aggregate suspension culture can be produced or utilized. Aggregate suspension culture methods include embryoid culture methods (see Keller et al., 1995), and a SFEB (serum-free embryoid body) methods (Watanabe et al., 2005); International Publication No. 2005/123902).

In certain aspects, non-static culture could be used for culturing and differentiation of pluripotent stem cells. The non-static culture can be any culture with cells kept at a controlled moving speed, by using, for example, shaking, rotating, or stirring platforms or culture vessels, particularly large-volume rotating bioreactors. In some embodiments, a rocker table may be used. The agitation may improve circulation of nutrients and cell waste products and also be used to control cell aggregation by providing a more uniform environment. For example, rotary speed may be set to at least or at most about 10, 15, 20, 25, 30, 35, 40, 45, 50, 75, 100 rpm, or any range derivable therein. The incubation period in the non-static culture for pluripotent stem cells, cell aggregates, differentiated stem cells, or progeny cells derived therefrom, may be at least or about 4 hours, 8 hours, 16 hours, or 1, 2, 3, 4, 5, 6 days, or 1, 2, 3, 4, 5, 6, 7 weeks, or any range derivable therein.

In some embodiments of pluripotent stem cell culturing, once a culture container is full, the colony is split into aggregated cells or even single cells by any method suitable for dissociation, which cells are then placed into new culture containers for passaging. Cell passaging or splitting is a technique that enables cells to survive and grow under cultured conditions for extended periods of time. Cells typically would be passaged when they are about 70%-100% confluent.

Single-cell dissociation of pluripotent stem cells followed by single cell passaging may be used in the present methods with several advantages, like facilitating cell expansion, cell sorting, and defined seeding for differentiation and enabling automatization of culture procedures and clonal expansion. For example, progeny cells clonally derived from a single cell may be homogenous in genetic structure and/or synchronized in cell cycle, which may increase targeted differentiation. Exemplary methods for single cell passaging may be as described in U.S. Pat. Publn. 2008/0171385, which is incorporated herein by reference.

F. Cryopreservation

The cells produced by the methods disclosed herein can be cryopreserved, see for example, PCT Publication No. 2012/149484 A2, which is incorporated by reference herein, at any stage of the process, such as Stage I, Stage II, or Stage III. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C. and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 6, 7, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8%, to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C./minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### III. METHODS OF USE

The present disclosure provides methods for producing iPSC-derived dopaminergic neurons with disease-associated mutations. These can be used for a number of important research, development, and commercial purposes. These include, but are not limited to, transplantation or implantation of the cells in vivo; screening cytotoxic compounds, carcinogens, mutagens, growth/regulatory factors, pharmaceutical compounds, etc., in vitro; studying the mechanism by which drugs and/or growth factors operate; diagnosing and monitoring cancer in a patient; gene therapy; and the production of biologically active products, to name but a few.

Midbrain DA precursors (e.g., D17 cells) provided herein can be used to screen for factors (such as solvents, small molecule drugs, peptides, and polynucleotides) or environmental conditions (such as culture conditions or manipulation) that affect the characteristics of DA neurons provided herein.

In some applications, stem cells (differentiated or undifferentiated) are used to screen factors that promote maturation of cells along the neural lineage, or that promote proliferation and maintenance of such cells in long-term culture. For example, candidate neural maturation factors or growth factors can be tested by adding them to stem cells in different wells, and then determining any phenotypic change that results, according to desirable criteria for further culture and use of the cells.

Cell cultures provided herein may be used, e.g., in testing the effect of molecules on neural differentiation or survival, or in toxicity testing or in testing molecules for their effects on neural or neuronal functions. This can include screens to identify compounds that affect neuron activity, plasticity (e.g., long-term potentiation), or function. The cell cultures may be used in the discovery, development and testing of new drugs and compounds that interact with and affect the biology of neural stem cells, neural progenitors or differentiated neural or neuronal cell types. The neural cells can also have great utility in studies designed to identify the cellular and molecular basis of neural development and dysfunction including but not limited to axon guidance, neurodegenerative diseases, neuronal plasticity and learning and memory. Such neurobiology studies may be used to identify novel molecular components of these processes and provide novel uses for existing drugs and compounds, as well as identify new drug targets or drug candidates.

In some applications, compounds can be screened or tested for potential neurotoxicity. Cytotoxicity can be determined in the first instance by the effect on cell viability, survival, morphology, or leakage of enzymes into the culture medium. In some embodiments, testing is performed to determine whether the compound(s) affect cell function (such as neurotransmission or electrophysiology) without causing toxicity.

In some embodiments, one or more specific compounds may be tested to determine if the compound has effects that may be beneficial for the treatment of a disease. Based on the effects of the compound on the functional activity, one may then be able to determine if the compound may be useful for the treatment of a disease. In some embodiments, the cells are derived from iPS cells from a subject that has a disease (e.g., a genetic disease or a disease with a genetic component or risk factor) such as a neurological or neurodegenerative disease (e.g., autism, epilepsy, ADHD, schizophrenia, bipolar disorder, etc.). In some embodiments, the cells may be cultured in the presence of a first compound or toxin so that the neural culture will display properties similar to a disease state; in these embodiments, a second compound may be provided to the cell cultures to see if the second compound can alleviate or reduce the effect of the first compound or toxin. In other embodiments, the cells cultures may be used to determine if a compound produces toxicity or adverse effects on the cell culture.

For example, one or more candidate agents may be added at varying concentrations to the culture medium. An agent that promotes the expression of a polypeptide of interest expressed in the cell is considered useful; such an agent may be used, for example, as a therapeutic to prevent, delay, ameliorate, stabilize, or treat an injury, disease or disorder characterized by a defect in neurodevelopment or neurological function. Once identified, agents may be used to treat or prevent a neurological condition. In another embodiment, the activity or function of a cell of the organoid is compared in the presence and the absence of a candidate compound. Compounds that desirably alter the activity or function of the cell are selected as useful in the present methods.

Agents useful in the present methods may be identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the present methods. Agents used in screens may include known those known as therapeutics for the treatment of neurological conditions. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

The assays to determine functional activity of the cells may comprise survival assays, GBA assays, calcium assays, MEA assays, synaptic pruning by microscopy assays, signal transduction chasing phosphorylated intermediates of various pathways, analysis of analytes released in the media in culture with normal and disease specific cell types. For example, for disease modeling applications where isogenically engineered or patient-specific cells are compared to AHN controls, the treatment or exposure to neurogenerative proteins like amyloid beta, myelin, synaptosomes or Tau would result in decreased calcium signaling and electrical activity as well as increased neuroinflammatory cytokines. For example, an increase (e.g., more than 30%, 40%, 50%, 60%, 70%, 80%, or 90%) in any of these functional activity measurements may indicate a candidate agent.

In some aspects, early pathogenic changes may be quantified by observing alterations or predominantly downregulation in gene expression profiles associated with the onset of neurodegeneration. In certain aspects, upregulation of immune related genes associated with the release of neuroinflammatory cytokines may be measured as associated with neurodegeneration.

The assays may be performed in a high-throughput manner. For example, the cell cultures can be positioned or placed on a culture dish, flask, roller bottle or plate (e.g., a single multi-well dish or dish such as 8, 16, 32, 64, 96, 384 and 1536 multi-well plate or dish), optionally at defined locations, for identification of potentially therapeutic molecules. Libraries that can be screened include, for example, small molecule libraries, siRNA libraries, and adenoviral transfection vector libraries. The screening platform may be automated, such as robotic automation. The culturing platform may comprise an automated cell washer and high content imager.

In some aspects, the present assay may quantify the response of the present cell cultures to different neuroinflammatory stimuli mimicking sterile bacterial infection (lipopolysaccharide (LPS) exposure), mechanical injury (scratch), and seizure activity (glutamate-induced excitotoxicity). The secreted cytokine profile of control and LPS-exposed cultures may be measured

The models as described herein may be used for compound or treatment screening or testing (e.g., for efficacy, toxicity, or other metabolic or physiological activity) for pharmacodynamic or pharmacokinetic testing of the passage of agents through the blood brain barrier, etc. Such testing may be carried out by providing a model as described herein under conditions which maintain constituent cells of that product alive (e.g., in a culture media with oxygenation); applying a compound to be tested (e.g., a drug candidate) to the cells (e.g., by administration to the endothelial layer); and then detecting a penetration of the compound through the endothelial layer and/or other physiological response (e.g., damage, scar tissue formation, infection, cell proliferation, burn, cell death, marker release such as histamine release, cytokine release, changes in gene expression, etc.), which may indicate whether said compound can penetrate the blood brain barrier and/or has therapeutic efficacy, toxicity, or other metabolic or physiological activity in the brain if systemically delivered (e.g., intravascularly) to a mammalian subject. A control sample of the model may be maintained under like conditions, to which a control compound (e.g., physiological saline, compound vehicle or carrier) may be applied, so that a comparative result is achieved, or damage can be determined based on comparison to historic data, or comparison to data obtained by application of dilute levels of the test compound, etc.

Methods of determining whether a test compound has immunological activity may include testing for immunoglobulin generation, chemokine generation and cytokine generation by the DA neurons of the cellular model.

Methods of crossing the blood brain barrier (e.g., the human blood brain barrier) that may be tested with the models taught herein include, but are not limited to, assessing permeability of different paracellular tight junctions, passive diffusion through the cell layers, receptor-mediated transcytosis, and/or cell efflux inhibition.

In some embodiments, the model may be used in personalized testing of a subject (e.g., for efficacy, toxicity, or other metabolic or physiological activity) for pharmacodynamic or pharmacokinetic testing of the passage of agents through the blood brain barrier, etc., with at least some of the cells of the model being from the subject. For example, fibroblast cells of the subject may be directed to induced pluripotent stem cells (e.g., induced pluripotent neural stem cells), which cells thereafter are directed to one or more cell types for the model, e.g., neuronal cells, oligodendrocytes, endothelial cells, astrocytes, or microglia.

The term “neurodegenerative disease or disorder” and “neurological disorders” encompass a disease or disorder in which the peripheral nervous system or the central nervous system is principally involved. The compounds, compositions, and methods provided herein may be used in the treatment of neurological or neurodegenerative diseases and disorders. As used herein, the terms “neurodegenerative disease”, “neurodegenerative disorder”, “neurological disease”, and “neurological disorder” are used interchangeably.

Examples of neurological disorders or diseases include, but are not limited to chronic neurological diseases such as diabetic peripheral neuropathy (including third nerve palsy, mononeuropathy, mononeuropathy multiplex, diabetic amyotrophy, autonomic neuropathy and thoracoabdominal neuropathy), Alzheimer's disease, age-related memory loss, senility, age-related dementia, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical basal degeneration, ALS-Parkinson's-Dementia complex of Guam, subacute sclerosing panencephalitis, Huntington's disease, Parkinson's disease, multiple sclerosis (“MS”), synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-Korsakoffs related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Other conditions also included within the methods of the present disclosure include age-related dementia and other dementias, and conditions with memory loss including vascular dementia, diffuse white matter disease (Binswanger's disease), dementia of endocrine or metabolic origin, dementia of head trauma and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including, but not limited to, epidural, subdural, subarachnoid, and intracerebral), and intracranial and intravertebral lesions (including, but not limited to, contusion, penetration, shear, compression, and laceration). Thus, the term also encompasses acute neurodegenerative disorders such as those involving stroke, traumatic brain injury, schizophrenia, peripheral nerve damage, hypoglycemia, spinal cord injury, epilepsy, and anoxia and hypoxia.

A. Pharmaceutical Compositions

Also provided herein are pharmaceutical compositions and formulations comprising the present cells and a pharmaceutically acceptable carrier.

Cell compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen.

Pharmaceutical compositions and formulations as described herein can be prepared by mixing the active ingredients (such as cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

A. Distribution for Commercial, Therapeutic, and Research Purposes

In some embodiments, a reagent system is provided that includes cells that exists at any time during manufacture, distribution or use. The kits may comprise any combination of the cells described in the present disclosure in combination with undifferentiated pluripotent stem cells or other differentiated cell types, often sharing the same genome. Each cell type may be packaged together, or in separate containers in the same facility, or at different locations, at the same or different times, under control of the same entity or different entities sharing a business relationship. Pharmaceutical compositions may optionally be packaged in a suitable container with written instructions for a desired purpose, such as the mechanistic toxicology.

In some embodiments, a kit that can include, for example, one or more media and components for the production of cells is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits of the present disclosure also will typically include a means for containing the kit component(s) in close confinement for commercial sale. Such containers may include injection or blow molded plastic containers into which the desired vials are retained. The kit can also include instructions for use, such as in printed or electronic format, such as digital format.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation and Characterization of iPSC-Derived DA Neurons

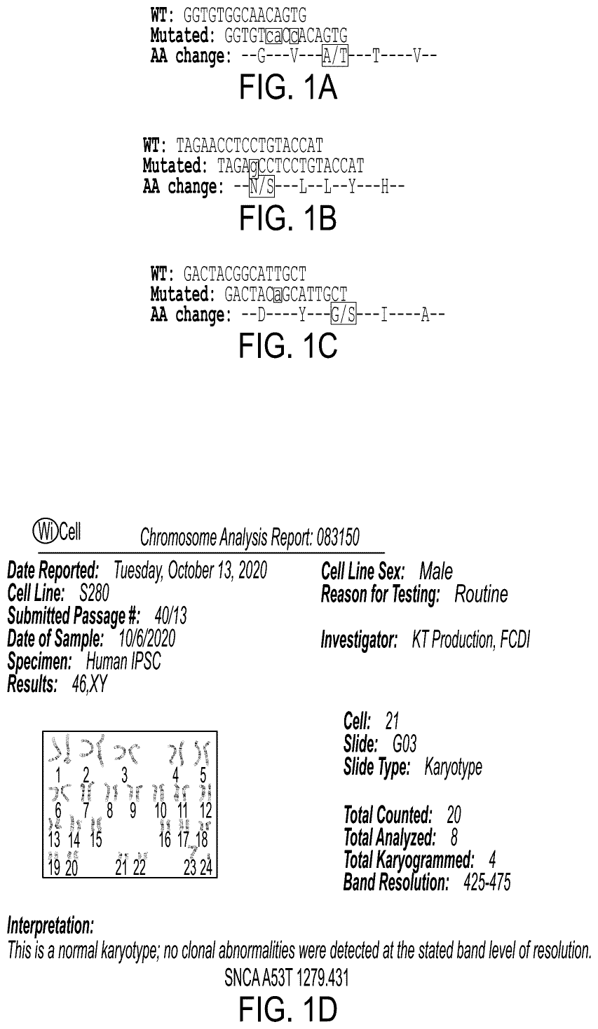

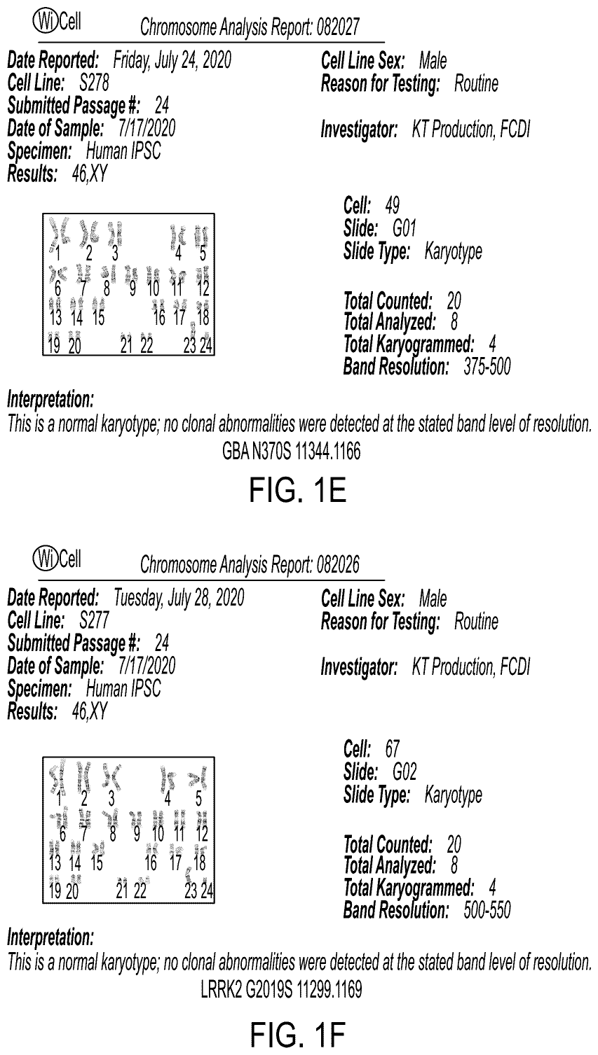

Episomally reprogrammed iPSC 01279 were engineered to generate a heterozygous (HZ) SNCA A53T mutant line (FIG. 1A). SNCA A53T HZ (SNCA A53T) iPSCs were genetically engineered from AHN iPSC 01279 by nuclease-mediated homologous recombination and a donor oligo SJD 14-132 (SEQ ID NO:1 GTTTTACAATTTCATAGGAATCTTGAATACTGGGCCACACTAATCACTAGATACT TTAAATATCATCTTTGGATATAAGCACAATGGAGCTTACCTGTgGtgACACCATGC ACCACTCCCTCCTTGGTTTTGGAGCCTACAAAAACAAATTCAAGACATAAGTCTC AAGCTAGCCTTAAATTGCTGATTAGCTAGTTTTC). The resulting iPSCs contained SNP rs104893877 where amino acid 53 was changed from alanine to threonine resulted in the A53T variant in the mutations resulting in the SNCA A53T Heterozygous iPSC line (SNCA A53T), provided a disease alpha-synuclein gene (SNCA) as well as two silent mutations for PD. The engineering is schematically described in FIG. 1B showing the amino acid 53 change from alanine to threonine highlighted in yellow.

Next, iPSC were derived from a PD patient harboring the GBA N370S mutation. The amino acid change of asparagine 370 to a serine is highlighted in yellow in FIG. 1B. iPSC were derived from a PD patient harboring the LRRK2 G2019S mutation which is schematically described, showing the amino acid change of glycine 2019 to a serine highlighted in yellow in FIG. 1C. Cytogenetic analysis on G-banded metaphase cells from each iPS cell line was performed detailing a normal karyotype, SNCA A53T, GBA (N370S), and LRRK2 (G2019S) (FIGS. 1D-1F). G-banding karyotyping was performed and analyzed by WiCell (Madison, WI).

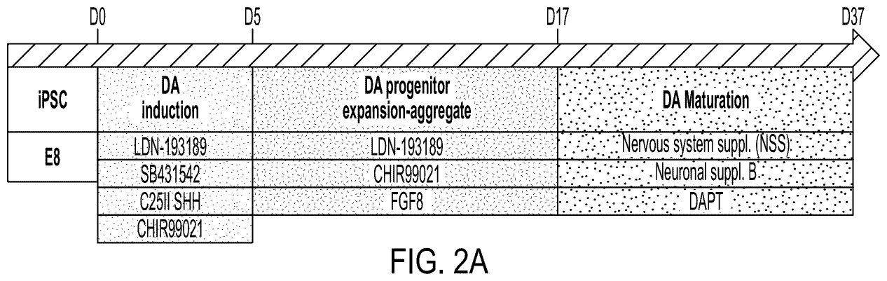

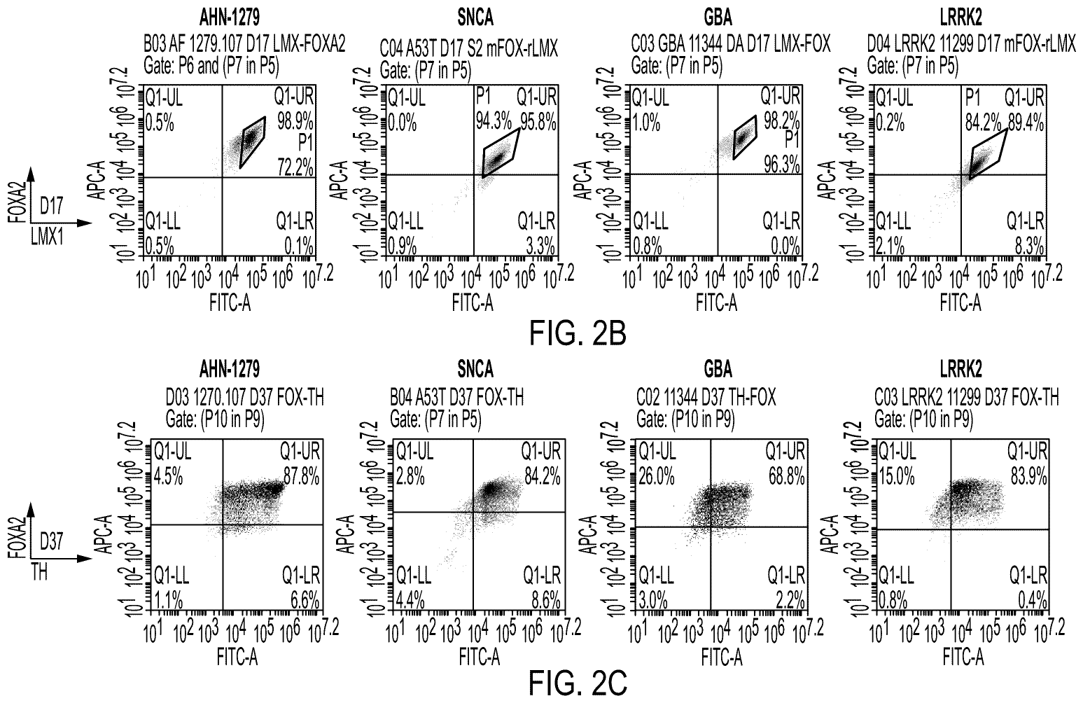

A schematic representation of the differentiation process from iPSC to the dopaminergic neurons with the timeline and cytokines utilized throughout the process is shown in FIG. 2A. Undifferentiated iPS were cultured feeder-free on MATRIGEL® coated plates and maintained using E8 medium (Thermo Fisher). Neuronal patterning started at DO by changing E8 medium and adding DMEM/F12 basal media [containing N2 (1×), B27 (−vitA) (1×) neuronal supplements (Gibco), LDN193189, SB431542, SHH, purmorphamine and CHIR99021]. At day 5, cells were dissociated and cultured in spinner flasks as aggregates. The cells were cultured in DMEM/F12 basal media [containing N2 (1×), B27 (−vitA) (1×) neuronal supplements (Gibco), LDN193189, CHIR99021 and FGF8] for 12 days. The cells were harvested at day 17 and assessed by staining for FOXA2 and LMX1 by flow cytometry. Cells were further matured in the neuronal maturation media (Neurobasal media+ Nervous system supplement and neural supplement B (FCDI) and DAPT (Tocris)) for 20 days and dissociated and cryopreserved at day 37. Representative flow cytometry dot plots of the dopaminergic progenitors at day 17, expressing midbrain transcription factors FOXA2 and LMX1 are shown in FIG. 2B. FIG. 2C shows representative flow cytometry dot plots of the dopaminergic neurons at the end of process expressing FOXA2 and tyrosine hydroxylase (TH) enzyme which is the enzyme needed for dopamine synthesis from tyrosine.

TABLE 1
Differentiation Media Compositions for DA Differentiation.

| | | | | |
|---|---|---|---|---|
| Component | Vendor | Cat# | Stock | Final Conc. |

E8 Medium

| | | | | |
|---|---|---|---|---|
| Essential 8 Basal Medium | Life Technologies | A14666SA | 1X | 98% |
| Essential 8 Supplement | Life Technologies | A14666SA | 50X | 2% |

DA Induction Medium

| DMEM/F12 | Life Technologies | 11330-032 | 1X | | 98% | |
|---|---|---|---|---|---|---|
| B-27 Supplement (+VitA) | Life Technologies | 17504-044 | 2X | | 2% | |
| LDN-193189 | Stemgent | 04-0074 | 10 | mM | 200 | nM |
| SB431542 | Sigma | S4317 | 40 | mM | 10 | μM |
| Purmorphamine | Cayman | 10009634 | 10 | mM | 2 | μM |
| C25II SHH | R&D Systems | 464-SH | 100 | μg/mL | 100 | ng/mL |
| CHIR99021 | Stemgent | 04-0004 | 20 | mM | 1.25 | μM |

DA Aggregate Formation Medium

| DMEM/F12 | Life Technologies | 11330-032 | 1X | | 98% | |
|---|---|---|---|---|---|---|
| B-27 Supplement (−VitA) | Life Technologies | 12587-010 | 2X | | 2% | |
| LDN-193189 | Stemgent | 04-0074 | 10 | mM | 200 | nM |
| Purmorphamine | Cayman | 10009634 | 10 | mM | 2 | μM |
| C25II SHH | R&D Systems | 464-SH | 100 | μg/mL | 100 | ng/mL |
| CHIR99021 | Stemgent | 04-0004 | 20 | mM | 1.25 | μM |

| | | | | | |
|---|---|---|---|---|---|
| Blebbistatin | Sigma | B0560 | 2,500x | 10 | μM |

Neuronal Maturation Medium

| Neurobasal | Life Technologies | 21103-049 | 1X | | 98% | |
|---|---|---|---|---|---|---|
| Glutamax | Life Technologies | 35050-061 | 200 | mM | 2 | mM |
| B-27 Supplement (−VitA) | Life Technologies | 12587-010 | 2X | | 2% | |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |

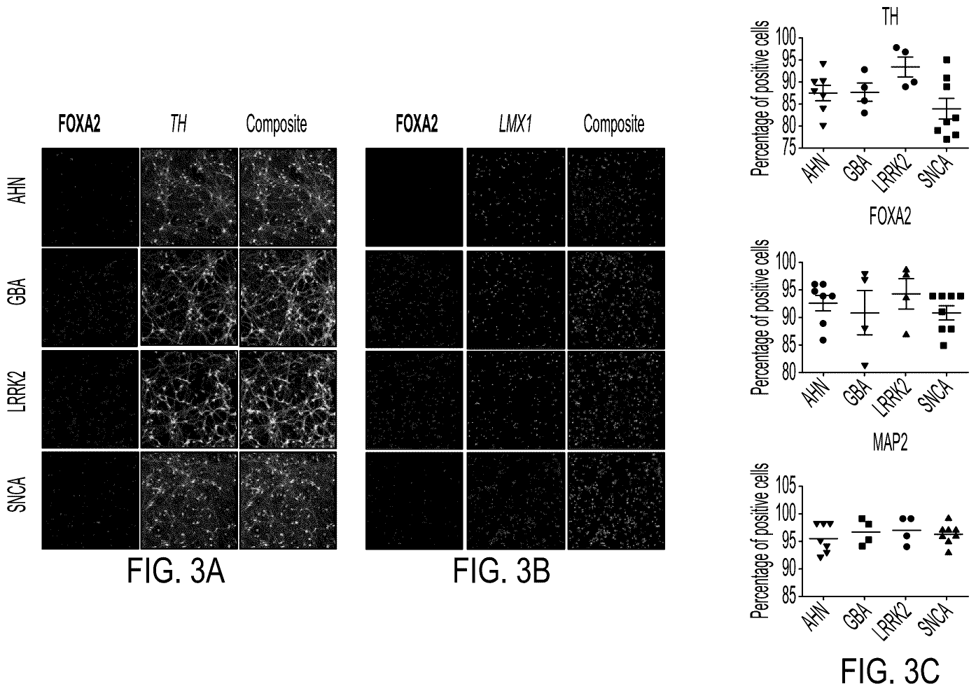

Immunofluorescent staining was performed on end stage DA neurons (FIGS. 3A-3B). Cryopreserved DA neurons were thawed and plated at 200 K/cm² in 96 well Greiner imaging plates. Cells were fixed at day 3 and 14 post thaw for 20 minutes with 4% paraformaldehyde in PBS. The cells were stained using fluorescein-conjugated secondary antibodies for one hour at room temperature, and counterstained with Hoechst for 20 minutes and washed. Samples were imaged on ImageXpress (Molecular Devices) (FIG. 3B). Flow cytometric analysis was performed for quantification of FOXA2 and TH proteins in dopaminergic neurons (FIG. 3C). Multiple lots of cryopreserved DA neurons from GBA, LRRK2, SNCA and AHN iPSCs were plated and harvested on day 3 and 14 using Accutase for 15 minutes at 37° C. Cells were stained with LIVE/DEAD™ fixable red dead cell stain kit (ThermoFisher) and then fixed for 20 minutes with 4% paraformaldehyde in PBS. The cells were stained for the presence of FOXA2 and TH expression and were quantified by intracellular flow cytometry.

GBA and LRRK2 patient iPSC-derived DA neurons showed aberrant mRNA and protein expression of enzymes involved in dopamine synthesis and degradation (FIG. 4A). TH, DDC, MAOA and COMT transcript levels were quantified in ANH, engineered and PD patient derived DA neurons. Multiple lots of cryopreserved DA neurons from GBA, LRRK2, SNCA and AHN iPSCs were plated and harvested on day 14 post thaw and the pellet was submitted for RNA Seq analysis. The quantification of TH, DDC, MAOA and COMT transcripts across multiple lots of DA neurons using the (FPKM) values was analyzed (FIG. 4B). TH and DDC protein levels were quantified in ANH, engineered and PD patient derived DA neurons. Multiple lots of cryopreserved DA neurons from GBA, LRRK2, SNCA and AHN iPSCs were plated and harvested on day 14 post thaw and cell lysates were loaded onto capillaries probed using TH (1:250, T2928 Sigma), GBA (1:100, R&D MAB4710), DDC (1:500, Novus Biologicals NB300-174), MAPT (1:1000, MAB361 Millipore) to detect the presence of TH and GBA proteins using the Protein Simple system according to the manufacturer's instructions. TH and DDC protein was quantified based on the area under the curve for each protein and normalized to the MAPT expression. (*P-value<0.05, **P-value<0.01, ***P-value<0.001) (FIG. 4C).

GBA and LRRK2 PD patient iPSC-derived DA neurons released more dopamine when stimulated with KCl (FIG. 5A). Multiple lots of cryopreserved DA neurons from GBA, LRRK2, SNCA and AHN iPSCs were thawed and cultured for 21 days and the levels of dopamine released by the cells were quantified using a competitive dopamine ELISA (Eagle Biosciences Cat. No. DOP31-KO1) following the manufacturer's instructions. Three technical replicates were performed per biological sample. The standard curve was graphed in Graphpad PRISM and depicted in FIG. 5A. The quantification of dopamine was calculated using non-linear regression using the log(agonist) vs. response-variable slope (four parameters) model. The dopamine release of DA neurons was compared to the dopamine released by AHN DA neurons at day 21 cultures in the HBSS buffer and upon KCL stimulation (FIG. 5B) (*P-value<0.05, **P-value<0.01, ***P-value<0.001).

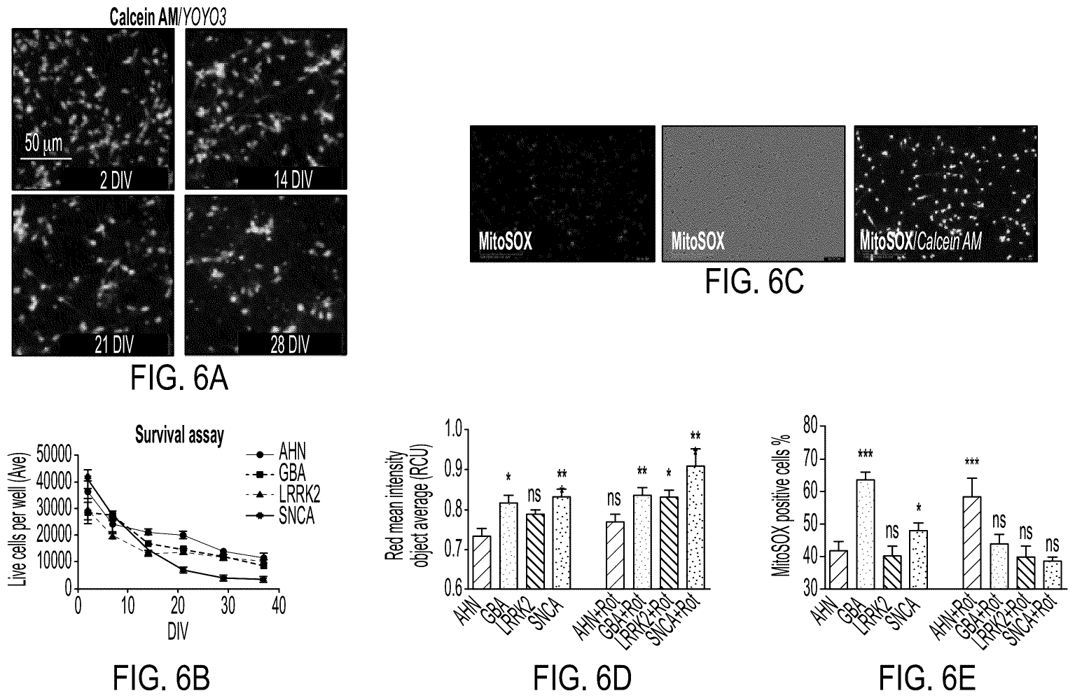

SNCA engineered iPSC and PD patient iPSC-derived DA neurons revealed higher cell death and mitochondrial stress (FIG. 6A). Multiple lots of cryopreserved DA neurons derived from GBA, LRRK2, SNCA and AHN iPSCs were thawed and plated. The survival rate over time and mitochondrial oxidative stress in the DA neurons was quantified by live culture staining with Calcein AM and YOYO-3 to quantify live and dead cells at different timepoints over the course of 38 days culture. Representative staining of end stage DA neurons at 21 days post thaw with 1 μM YOYO-3 Iodide and Calcein AM is shown in FIG. 6A. Multiple lots of cryopreserved DA neurons derived from GBA, LRRK2, SNCA and AHN iPSCs were thawed and plated in 96 well optical plates at a density of 64,000 cells per well. The cells were incubated with 1 μM YOYO-3 Iodide and 1 μM calcein AM (Thermo Fisher) for 30 min at RT and imaged in the Incucyte S3 for 3 hours every 30 minutes (FIG. 6B). The percentage of live/dead cells was calculated based on the positive objects in the green and red channels. The cells were washed with PBS and lysed in the CellTiter-Glo cell viability assay buffer and incubated for 10 minutes following 1 minute of shaking. Luminescence signal was captured and quantified using CLARIOstar plate reader (Molecular Devices). The data depicts live cells per well of 96 well plate (0.143 cm²). FIG. 6C shows representative images of DA neurons stained with MitoSOX dye (left image) and overlay with the phase image (Middle image) and co-stained with Calcein AM for live cells (right image).

Multiple lots of cryopreserved DA neurons derived from GBA, LRRK2, SNCA and AHN iPSCs were thawed and plated. MitoSOX red (5 μM) was added to the neurons and incubated for 30 minutes at 37° C. MitoSOX was then removed and Calcein AM (Thermo Fisher, C1430) was added to the neurons and imaged in the Incucyte S3 every 30 minutes for 3 hours. Rotenone (mitochondrial toxin) at 2 μM was used as a positive control. Neurons for positive control were treated with Rotenone for 30 mins at 37° C. followed by a wash with the complete neuronal medium. The quantification of the MitoSOX signal intensity (Means±SD) and percentage of the MitoSOX positive cells in the disease neurons compared to the AHN neurons was determined and the results were compared against the ROS in the mitochondria induced by rotenone (FIGS. 6D-6E).

SNCA engineered iPSC and PD patient derived DA neurons had significantly more aSyn protein aggregation (FIG. 7). Alpha-synuclein protein aggregation was observed in the DA neurons. Thioflavin (Th-T) staining was performed to reveal the aggregated aSyn on live DA neurons culture at 22 DIV after adding oligomer aSyn (4 μM/ml) for 24 hrs (FIG. 7A). All the aSyn⁺ cells were counted and plotted against Th-T signal intensity in the individual neurons in different bins of expression (FIG. 7B). Th-T images were quantified on day 21 culture treated with active oligomer aSyn for 24 hrs, 48 hrs or 72 hrs (n=4, One-way ANOVA with Dunnett's multiple comparisons test) (FIG. 7C). For the Alpha-Synuclein protein aggregation assay (Th-T staining), cells were seeded at the density of 64K per well in the 96 well plate for Th-T staining. At day 21, post thaw cells were treated with 4 ug/ml of recombinant mouse Alpha-synuclein protein aggregate (Active) (Abcam, ab246002) for 24, 48, and 72 hours and then stained with Thioflavin T. Thioflavin T (Th-T) (Abcam, ab120751) was dissolved in HBSS (Th-T dissolved completely at concentration of 8 mg/ml (2 mM) at 37° C.). Cells were stained for Th-T dye (10 uM final concentration) and YOYO-3 iodide (Thermofisher-Y3606 1 uM) in the culture media. Cells were incubated for 30 minutes, washed using warm HBSS and imaged using Incucyte 20× objective. The intensity of the signal for Th-T was quantified in each neuron for different conditions using Incucyte software.

SNCA engineered iPSC and PD patient iPSC-derived DA neurons had greater aSyn aggregation (FIG. 8). Quantification of Alpha-Synuclein protein aggregation was performed using the Meso Scale Diagnostics (MSD) U-PLEX human alpha-Synuclein kit (MSD-K15). Multiple lots of cryopreserved DA neurons derived from GBA, LRRK2, SNCA and AHN iPSCs were thawed and plated for 21(FIG. 8B) or 42 days (FIG. 8C) in culture. The cells were lysed in RIPA lysis buffer (Cell Signaling) supplemented with Halt Protease and Phosphatase inhibitor cocktail (Thermo Fisher Scientific) and the protein content was measured using Pierce BCA protein assay (Thermo Fisher Scientific). The quantification of human alpha-Synuclein was performed using the MSD-K15 kit from MSD. Multiple lots of cryopreserved DA neurons derived from GBA, LRRK2, SNCA and AHN iPSCs were thawed and cultured up to 42 days. Analyte concentrations were determined from the ECL signals by backfitting to the calibration curve (FIG. 8A). The calculations to establish calibration curves and determine concentrations were carried out using the MSD DISCOVERY WORKBENCH® analysis software. Alpha-Synuclein protein concentration for AHN was 34 ng/ml±0.16, versus SNCA=66.5 ng/ml±2.44, GBA=89.5 ng/ml±4.45 and LRRK2=53 ng/ml±0.68 (Mean±SD). Any alpha-Synuclein protein concentration increase above 20% of AHN cells could be considered as disease state.

SNCA engineered iPSC and PD patient iPSC-derived DA neurons were observed to have less GCase activity. Multiple lots of cryopreserved DA neurons derived from GBA, LRRK2, SNCA and AHN iPSCs were thawed and plated for 14 days and the samples were harvested and submitted for RNASeq analysis as well as western blot analysis. The FPKM values of the GBA transcript were quantified (FIG. 9A). Total cell lysates from all samples containing 2.5 ug of protein were loaded in gel capillaries using the Protein Simple instrument and the detection of 66 kd GBA protein was confirmed. The amount of GBA protein in the lysate was quantified based on the area under the curve for each protein and normalized to the MAPT expression (FIG. 9B). Different concentrations of 4-Methylumbelliferyl-β-D-glucopyranoside (4-MU) (FIG. 9C) as the substrate and GCase specific inhibitor Conduritol B Epoxide (CBE) (FIG. 9D) were tested. 10 mM of 4-MU and 2 mM of CBE were found to be the optimal concentrations and used for the following GCase activity assay. Different concentrations of protein lysate were also tested for the GCase activity from AHN DA neurons (FIG. 9E). 5 ug of protein lysate was used to compare GCase activities among different DA neurons (FIG. 9F). (n=4, p<0.001 one-way ANOVA with Dunnett's test for mean comparisons). (*P-value<0.05, **P-value<0.01, ***P-value<0.001).

For the GCase activity assay, multiple lots of cryopreserved DA neurons derived from GBA, LRRK2, SNCA and AHN iPSCs were thawed and plated for 14 days. The cells were harvested, lysed and centrifuged at 20,000×g for 20 min at 4° C. Total protein concentrations were measured using Pierce BCA protein assay (Thermo Fisher Scientific). 5 ug of protein per lysate was used to determine GCase activity in the presence of 1% BSA, with 10 mM 4-Methylumbelliferyl O-glucophyranoside (4-MU, Sigma-Aldrich, #M3633) (FIG. 9C) with or without 2 mM conduritol-b-epoxide (CBE) (FIG. 9D). Recombinant human Glucosylceramidase/GBA (rhGBA) (R&D, 7410-GHB-020) was used as a positive control. Samples were incubated for 40 minutes at 37° C. and reaction stopped by adding equivolume 1M glycine, pH 12.5. 100 μl of reactions were loaded into white 96-well plates (Nunc, #136101) and fluorescence (ex=355 nm, em=460, 0.1 s) signal were measured in the CLARIOstar plate reader (Molecular Devices). Relative fluorescent units (RFUs) from non CBE-treated lysates were calculated by subtracting the signal from the corresponding CBE-treated lysates. AHN cells had higher GCase activity compared to the mutants (mean±SD, AHN=31117±639, GBA=13622±385, LRRK2=20619±179 and SNCA=173429±29300).

SNCA engineered iPSC and PD patient iPSC-derived DA neurons revealed differences in the evolvement of neural network activity (FIG. 10). Multiple lots of cryopreserved DA neurons derived from GBA, LRRK2, SNCA and AHN iPSCs were thawed and plated on the 48-well Classic multi-electrode array (MEA) plate (8 wells per lot at a cell density of 120K cells per well) in the complete BrainPhys media. Neuronal activity was quantified using the Axion Maestro MEA. MEA recordings were taken every few days to monitor the development of synchronously bursting neuronal networks in culture over time. The .RAW data files were converted to .spk files and subsequently analyzed using the Neural Metric Tool software from Axion. Burst detection was performed using the “envelope method”. Spontaneous action potentials (black tick marks are “spikes”), and bursting behavior (blue-color tick marks) was observed. Synchronous network bursts were also detected (see histogram peaks and corresponding pink/magenta boxes around the organized spikes below the bursts across all electrodes). The results revealed lower activity and network strength in GBA, LRRK2 and SNCA neurons compared to the AHN cells on day 35 raster plots (FIG. 10A) along with the quantification of burst percentage, network burst frequency and synchrony index over time (FIG. 10B).

For RNA-Seq analysis, dopaminergic neurons were thawed and maintained according to the iCell Dopa Neuron User's Guide. In brief, 2.0E+6 cells are plated onto PLO/Laminin coated 6-well tissue culture plates and cultured to more than 14-day post-thaw. Cells were lysed directly in the well using 400 microliters of Qiagen RLT+ lysis buffer. Duplicate samples/lot of ˜2 million cells each were lysed and stored at −80° C. until RNA extraction was performed. The QiaSymphony was used to extract the RNA. RNA concentration should be greater than 20 ng/μL and the ratio of absorbance (OD 260/280) should be greater than 2.0 to ensure acceptable purity of the sample.

cDNA library preparation was performed in Novogene, and samples were tailed and ligated with modified oligos, size selected and amplified. All the libraries were then tested prior to sequencing using 1) the Agilent BioAnalyzer for sizing and quality assessment of the library and 2) qPCR for measuring the concentration of the library and presence of Illumina anchor sequences. RNA sequencing was then performed on Illumina NovaSeq 6000 platform using paired-end technology with a read length of 150 base pairs. The output data files called “fastq” files consisting of >=20 million read pairs per sample were then shipped to FCDI on a hard drive. These raw data files were copied on to the linux cluster and archived after checking the integrity of the files. In-silico sample QC to check for possible sample swap was performed by aligning the reads using an alignment program called bowtie2 to the in-house sequence database consisting of human DNA and mRNA, mouse, phiX, viral, fungi and bacterial sequences. Samples were considered passed if they contained more than 75% of human sequences. The sequencing reads quality report was generated using the FASTQC program. All the high-quality reads were then aligned to the HG38 transcriptome using splice-aware alignment program, HISAT2. The alignment files (called bam files) were then used to generate read count matrices using Rsubread::FeatureCounts program that could be further used to run differential expression analysis between samples of interest using DESeq2 program. The normalized read counts were converted to log 10 (FPKM+0.0001) values and simple scatter plots could be visualized in TIBCO Spotfire software.

Transcripts of mutant DA neurons were plotted against the AHN DA neurons and showed a high correlation in the gene expression level with significantly differential transcripts highlighted (FIG. 11A). Differential gene expression analysis was carried out for mutant samples compared to the AHN samples and the number of transcripts that changed significantly was summarized in the Venn diagram (FIG. 11B). Gene set enrichment and pathway analysis for transcripts that were differentially regulated in mutant DA neurons compared to the AHN DA neurons in DAVID database are shown in FIG. 11C.

For compound screening, 12 compounds that were previously shown to have positive effects on improving GCase activity were selected. DA neurons were cultured at high density (200,000/well) in the 96 well plates. Gcase activity was measured from DA neurons derived from GBA, LRRK2 and AHN iPSCs after compound treatment. On day 18, post-thaw cells were dosed with different compounds at three concentrations (1×, 10× and 100× of compounds in Table 2). On day 21 post-plating (3 days post-dosing) cells were lysed using 100 ul of assay buffer and Gcase activity determined using 5 ug of protein lysate. Net Gcase activity was graphed in comparison to the 0.1% DMSO treated cells as the negative control. Total protein in the lysate was quantified using Pierce BCA Protein Assay Kit (ThermoFisher).

GCase activity can be used as a read out for compound screening with disease neurons where AHN serve as a control and compounds that could rescue the GCase activity would be considered a positive hit. Effects of 12 compounds at three concentrations on Gcase activity in GBA and LRRK2 DA neurons with AHN as the control are shown in FIG. 12A. For the combinatorial screen, 4 molecules were picked from initial screen showing improved GCase activity and combined in different ways to test with GBA cells (FIG. 12B). Single compounds were tested both for acute exposure (3 days) and chronic exposure (two weeks). Combinatorial screen showed that chronic treatment with 10 uM Ambroxol had the highest GCase activity in GBA cells (mean±SD, DMSO=90302±3986 compared to ABX=133659±10542, 48% increase in GCase activity). This was further tested in LRRK2, SNCA and GBA in comparison to AHN (FIG. 12C). At day 21 post thaw, treatment with Ambroxol at 10 uM for two weeks significantly increased Gcase activities in all mutant neurons. (Mean, AHN from 165052 to 171669, GBA from 108417 to 121225 (12% increase), LRRK2 from 152793 to 180112 (18% increase) and SNCA from 165107 to 189082 (14% increase)) (Dotted lines indicate the Gcase levels of the DM0 vehicle control, ***P-value<0.001.).

TABLE 2
Screened Compounds.

| | | Concentration |
|---|---|---|
| Name | Solvent | (1X) |
| α-1-C-Nonyl-DIX (N- | DMSO (100 mM), | 1 μM |
| Nonyldeoxynojirimycin)* | methanol, and ethanol | |
| | (100 mM). | |
| Ambroxol (ABX)* | DMSO (200 mg/ml) | 1 μM |
| NCGC758 | DMSO (2.5 mg/ml) | 1 μM |
| NCGC607 | DMSO (25 mg/ml) | 1 μM |
| Eliglustat (EGT) | DMSO (80 mg/ml) | 1 μM |
| KU-600 | DMSO (50 mg/ml) | 0.1 μM |
| Ropinirole | PBS(10 mg/ml) | 1 μM |
| (hydrochloride) | | |
| Echinacoside* | DMSO (30 mg/ml) | 1 μM |
| ML198 | DMSO 4 mg/mL | 1 μM |
| | (13.78 mM) | |
| Kifunensine | Water (0.5 mg/ml) | 1 μM |
| ELN484228 (NSC | DMF, DMSO | 1 μM |
| 164389) | (1 mg/ml) | |
| Methyl-β-cyclodextrin* | PBS: 10 mg/ml, | 100 μM |
| | DMSO: 30 mg/m | |

For calcium imaging experiments, DA neurons were cultured as spheroids (50,000/well) in the 384 U bottom ULA plates (S-bio plates) and fed with Brainphys complete media (Brainphys basal media+neural supplement A+NSS). On day 7 post-plating some of the wells were treated using 10 uM Ambroxol. On day 14 post-plating (7 days post-treatment) cells were loaded with 1× EarlyTOX cardiotoxicity kit and after 2 hours of incubation at 37° C., Ca2+ oscillation captured using FDSS/μCELL instrument for 20 minutes. Waveform software was used for quantification of Ca oscillation and data were analyzed using GraphPad Prism.

Calcium oscillation traces of day 14 post plated DA neurons were captured using FDSS/μCELL instrument for 20 minutes. Baseline readings of Ca oscillation in AHN and all three mutations after treatment with 10 uM Ambroxol for one week are shown in FIG. 13A. Compared to the untreated AHN DA neurons, treatment with Ambroxol caused hyperexcitability in AHN, LRRK2 and SNCA DA neurons but rescued the peak number and rate in GBA mutant DA neurons. Calcium transient properties including (FIG. 13B) number of peaks, (FIG. 13C) peak rates per minute and (FIG. 13D) area under the curve/peak (average) were quantified for all wells (repeats) of each cell line using Waveform software. Number of the peaks varies among mutations (disease neurons) and compared to the AHN (mean±SD, AHN=21.55±3, GBA=13.43±1, LRRK2=28.83±3.65 and SNCA=24.5±5.3). Ambroxol treatment increased the peak number for all neurons including the AHN (mean±SD, AHN=33.75±7.14, GBA=21.8±1.8, LRRK2=35.3±3 0.9 and SNCA=33.8±1.5). Peak rate which is number of peaks per minute also showed the difference among cell lines (mean±SD, AHN=1.12±0.16, GBA=0.72±0.06, LRRK2=1.52±0.16 and SNCA=1.26±0.3) and when treated with ambroxol it also increased (mean±SD, AHN=1.74±0.32, GBA=1.13±0.1, LRRK2=1.89±0.18 and SNCA=1.61±0.12). The area under the curve which is a direct measurement for how long the calcium channels are open and/or the cytosolic calcium, is also different in the mutant neurons compared to the AHN (mean±SD, AHN=34.6E04±13.5E04, GBA=57.3E04±13.4E04, LRRK2=27.4E04±10.3E04 and SNCA=41.3E04±17.6E04) and ambroxol treatment decreased the cytosolic calciumin GBA and SNCA but not in LRRK2 mutant DA neurons (mean±SD, AHN=25.7E04±13.0E04, GBA=38E04±78E03, LRRK2=26.1E04±49.5E03 and SNCA=37.4E04±41.7E03). Peak rate which is number of peaks per minute for normal healthy cells is 0.96<AHN<1.3 at day 14 post plating and 30% change considered as abnormal peak rate. The average area under the curve for healthy neurons is between 21.1E04<AHN<48.2E04 and above 40% change in area under the curve considered as abnormal peak. Peak rate and area under the curve (peak) can be used as read outs for compound screening with disease neurons when AHN serve as a control and compounds that could rescue those two parameters could be considered as positive hits in compound screening.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Andrews et al., In: Teratocarcinomas and Embryonic Stem Cells, Robertson (Ed.), IRL Press, 207-246,1987.
Chen et al., Nature Methods 8:424-429, 2011.
Evans et. al., Nature, 292:154, 1981.
Fernandes, et al., J. Biotechnology, 132(2):227-236, 2007.
Gaj et al., Trends in Biotechnology, 2013, 31(7), 397-405
Gene Targeting, A Practical Approach, IRL Press at Oxford University Press, 1993.
International Patent Publication No. 2012/149484 A2
International Patent Publication No. 98/30679
International Patent Publication No. PCT/US2011/046796
International Patent Publication No. WO 02/016536
International Patent Publication No. WO 03/016496
International Patent Publication No. WO 2009/149233
International Patent Publication No. WO 2010/141801
International Patent Publication No. WO 2011/091048
International Patent Publication No. WO 98/30679
International Patent Publication No. WO 98/53058
International Patent Publication No. WO 98/53059
International Patent Publication No. WO 98/53060
International Patent Publication No. WO01/088100
International Patent Publication No. WO2005/080554
International Patent Publication No. WO2005/123902
International Patent Publication No. WO2011130675
International Patent Publication No. WO2012080248
International Patent Publication No. WO2013/067362
International Patent Publication No. WO2013067362
International Patent Publication No. WO2013163228
Jainchill et al., J. Virol., 4:549, 1969.
Kodama et al., J. Cell. Physiol., 112:89, 1982.
Ludwig et al., Nat. Biotechnol., 24(2):185-187, 2006b.
Ludwig et al., Nat. Methods, 3(8):637-46, 2006a.
Manipulating the Mouse Embryo A Laboratory Manual, Second Edition, Cold Spring Harbor Laboratory Press, 1994.
Martin, Proc. Natl. Acad. Sci. USA, 78:7634, 1981.
Nakano et al., Science, 272, 722, 1996.
Takahashi and Yamanaka, Cell, 126:663-676, 2006.
Takahashi et al., Cell, 131:861-872, 2007.
U.S. application Ser. No. 13/054,022
U.S. Pat. No. 8,252,586
U.S. Pat. No. 8,426,200
U.S. Pat. No. 6,140,081
U.S. Pat. No. 6,453,242
U.S. Pat. No. 6,534,261
U.S. Pat. No. 7,820,439
U.S. Pat. No. 8,153,428
U.S. Patent Publication No. 2011/0053207
U.S. Patent Publication No. 2014/0038279
U.S. Patent Publication No. 2002/0076747
U.S. Patent Publication No. 2003/0211603
U.S. Patent Publication No. 2005/0064474
U.S. Patent Publication No. 2006/0188987
U.S. Patent Publication No. 2007/0116680
U.S. Patent Publication No. 2007/0218528
U.S. Patent Publication No. 2007/0238170
U.S. Patent Publication No. 2008/0171385
U.S. Patent Publication No. 2008/0171385
U.S. Patent Publication No. 2011/0301073
U.S. Patent Publication No. 2011/0301073
U.S. Patent Publication No. 2012/0276063
U.S. Patent Publication No. 2012/0276063
Watanabe et al., Nature Neurosci., 8:288-296, 2005.
Yakubov et al., Biochemical and Biophysical Research Communications 394: 189-193, 2010.
Yu and Thomson, Genes Dev. 22(15):1987-97, 2008.
Yu et al., Science, 318:1917-1920, 2007.
Yu et al., Science, 324(5928):797-801, 2009.

1. An isolated induced pluripotent stem cell (iPSC)-derived dopaminergic (DA) neuron cell line comprising a glucosylceramidase (GBA) mutation, leucine rich repeat kinase 2 (LRRK2) mutation, or alpha-Synuclein (SNCA) mutation.

2. (canceled)

3. (canceled)

4. The cell line of claim 1, wherein the cell line has GBA mutation selected from the group consisting of a GBA N370S mutation, GBA L444P mutation, or GBA RecNil mutation.

5. (canceled)

6. The cell line of claim 1, wherein the cell line has a LRRK2 mutation.

7. The cell line of claim 6, wherein the cell line has a LRRK2 G2019S mutation, LRRK2 R1441G mutation, LRRK2 R1441C mutation, or LRRK2 12020T mutation.

8. (canceled)

9. The cell line of claim 1, wherein the cell line has a SNCA mutation.

10. The cell line of claim 9, wherein the cell line has a SNCA A53T mutation, SNCA E46K mutation, SNCA duplication, or SNCA triplication.

11. (canceled)

12. The cell line of claim 1, wherein the iPSC of the iPSC-derived DA neuron cell line is an iPSC episomally reprogrammed from a donor with a neurodegenerative disease.

13. The cell line of claim 1, wherein the iPSC of the iPSC-derived dopaminergic neuron cell line is an iPSC episomally reprogrammed from a donor with Parkinson's disease.

14. The cell line of claim 1, wherein the iPSC of the iPSC-derived dopaminergic neuron is genetically engineered to comprise a GBA mutation, LRRK2 mutation, or SNCA mutation.

15. The cell line of claim 1, wherein the cell line is a human cell line.

16. The cell line of claim 1, wherein the iPSC-derived dopaminergic neurons are midbrain dopaminergic neurons.

17. The cell line of claim 1, wherein the iPSC-derived dopaminergic neurons are end stage dopaminergic neurons which express FOXA2 and tyrosine hydroxylase (TH).

18. The cell line of claim 1, wherein the iPSC-derived dopaminergic neurons have at least 50% increased transcript levels of TH, DDC, MAOA, and/or COMT as compared to DA neurons differentiated from iPSCs reprogrammed from a healthy donor without disease-associated mutations.

19. The cell line of claim 1, wherein the iPSC-derived dopaminergic neurons have at least 30% increased release of dopamine in response to KCl stimulation as compared to DA neurons differentiated from iPSCs reprogrammed from a healthy donor without disease-associated mutations.

20. The cell line of claim 1, wherein the iPSC-derived dopaminergic neurons have increased cell death, mitochondrial stress, and alpha-synuclein protein aggregation as compared to DA neurons differentiated from iPSCs reprogrammed from a healthy donor without disease-associated mutations.

21. The cell line of claim 1, wherein the cell line is isogenic.

22. The cell line of claim 1, wherein the iPSC-derived dopaminergic neurons comprising an SNCA mutation have decreased lysosomal GCase activity as compared to DA neurons differentiated from iPSCs reprogrammed from a healthy donor without disease-associated mutations.

23. (canceled)

24. The cell line of claim 1, wherein the iPSC-derived dopaminergic neurons have deficits in intracellular calcium signaling as measured by RNA sequencing and calcium imaging.

25. A kit comprising the cell line of claim 1 in a suitable container.

26. The kit of claim 25, wherein the kit comprises an iPSC-derived DA neuron cell line comprising a GBA mutation in a first container and an iPSC-derived DA neuron cell line comprising a LRRK2 mutation in a second container.

27-34. (canceled)

35. The kit of claim 25, further comprising astrocytes, pericytes, brain microvascular endothelial cells, microglia, and/or neurons each in a suitable container.

36-44. (canceled)

45. A culture comprising iPSC-derived DA neurons comprising a GBA mutation, a LRRK2 mutation, or a SNCA mutation.

46-93. (canceled)
